(19) United States
(12) Patent Application Publication
(10) Pub. No.: US 2011/0081362 A1
(43) Pub. Date: Apr. 7, 2011

(54) TREATMENT OF CANCER

(75) Inventors: Stephen J. Elledge, Brookline, MA (US); Ji Luo, Allston, MA (US); Michael Schlabach, Newton, MA (US); Nicole Solimini, Quincy, MA (US)

(73) Assignee: THE BRIGHAM AND WOMEN'S HOSPITAL, INC., Boston, MA (US)

(21) Appl. No.: 12/865,564

(22) PCT Filed: Feb. 2, 2009
(86) PCT No.: PCT/US09/32808

§ 371 Date: Dec. 10, 2010

Related U.S. Application Data
(60) Provisional application No. 61/025,067, filed on Jan. 31, 2008.

(51) Int. Cl. A61K 39/395; A61K 31/7105; A61K 31/7088; A61K 38/06; A61K 31/4184; A61K 31/337; A61K 31/505; A61K 31/4985; C12Q 1/02; A61P 35/00

(52) U.S. Cl. 424/174.1; 514/44 A; 514/44 R; 514/19.3; 514/395; 514/449; 514/274; 514/249; 435/29

(57) ABSTRACT

Described herein are methods based, in part, on the discovery of genes or gene products that can be down-modulated to inhibit the growth and survival of a cell, such as a cancer cell. In one embodiment, the genes or gene targets are preferentially expressed in a cell having an activating Ras mutation (e.g., a cancer cell), which permits selective inhibition of growth in cells bearing an activating Ras mutation without affecting cells lacking enhanced Ras activity. In addition, the methods described herein provide for determining cancer prognosis in an individual bearing an activating Ras mutation.

### BACKGROUND OF THE INVENTION

Despite years of research into the development of new methods of treatment, many types of cancer, including, e.g., breast and colon cancer, remain quite common. There is a clear need for more anti-cancer agents to complement or replace existing treatments.

### SUMMARY OF THE INVENTION

The methods described herein are based, in part, on the discovery of genes or gene products that can be down-modulated to inhibit the growth and survival of a cell, such as a cancer cell. In one embodiment, the genes or gene targets are preferentially expressed in a cell having an activating Ras mutation (e.g., a cancer cell), which permits selective inhibition of growth in cells bearing an activating Ras mutation without affecting cells lacking enhanced Ras activity. In addition, the methods described herein provide for determining cancer prognosis in an individual bearing an activating Ras mutation.

In one aspect, the methods described herein are useful for inhibiting growth or survival of a cancer cell bearing a Ras mutation, the method comprising contacting a cancer cell bearing a Ras mutation with an inhibitor of at least one gene from Table 2, wherein the inhibitor reduces growth or survival of the cancer cell.

In one embodiment of this aspect and all other aspects described herein, the inhibitor disrupts mitosis in the cancer cell.

In another embodiment of this aspect and all other aspects described herein, the inhibitor is selected from the group consisting of an RNA interference molecule, a small molecule, an antibody, an aptamer and a nucleic acid.

In another embodiment of this aspect and all other aspects described herein, the method further comprises activation of APC/C activity.

In another embodiment of this aspect and all other aspects described herein, the contacting step comprises treating the cancer cell with an inhibitor of a plurality of the genes.

In another embodiment of this aspect and all other aspects described herein, the method further comprises contacting with a chemotherapeutic agent for combination therapy.

In another embodiment of this aspect and all other aspects described herein, the inhibitor targets a regulator of mitosis or chromosomal segregation.

In another embodiment of this aspect and all other aspects described herein, the cancer cell is in culture.

In another embodiment of this aspect and all other aspects described herein, the regulator is a gene product selected from the group consisting of cyclin A2 (CCNA2), hMIS18α, hMIS18β, C21ORF45, OIP5, borealin (CDCA89), KNL-1 (CASC5), MCAK (KIF2C), subunits of the APC/C complex (ANAPC1, ANAPC4, CDC16, CDC27), SMC4, and the mitotic kinase PLK-1.

In another embodiment of this aspect and all other aspects described herein, the inhibitor is a small molecule selected from the group consisting of paclitaxel, nocodazole, monastrol, and BI-2536.

In another embodiment of this aspect and all other aspects described herein, the inhibitor is selected from the group consisting of those listed in Table 7 or 8.

In another embodiment of this aspect and all other aspects described herein, the inhibitor targets a regulator of APC/C or proteasomes.

In another embodiment of this aspect and all other aspects described herein, the regulator is a gene product selected from the group consisting of APC1/ANAPC1, APC4/ANAPC4, Cdc16, Cdc27, PSMA5, PSMB5, and PSMB6.

In another embodiment of this aspect and all other aspects described herein, the inhibitor is selected from the group consisting of those listed in Table 7 or 8.

In another embodiment of this aspect and all other aspects described herein, the method further comprises activating EMI™, or EV15 activity.

In another embodiment of this aspect and all other aspects described herein, the method further comprises combination treatment of the individual with BI-2536.

In another embodiment of this aspect and all other aspects described herein, the inhibitor is MG132, or bortezomib.

Also described herein is a method for treating cancer in an individual, the method comprising: (a) determining whether cancer cells of the individual bear an activating Ras mutation; and, if so, (b) administering to the individual an inhibitor of at least one gene selected from Table 2, wherein the inhibitor treats cancer in the individual.

In one embodiment of this aspect and all other aspects described herein, the inhibitor is selected from the group consisting of an RNA interference molecule, a small molecule, an antibody, an aptamer and a nucleic acid.

In another embodiment of this aspect and all other aspects described herein, the method further comprises activation of APC/C activity.

In another embodiment of this aspect and all other aspects described herein, the cancer is breast cancer or colon cancer.

In another embodiment of this aspect and all other aspects described herein, the administering step further comprises administering an inhibitor of a plurality of the genes.

In another embodiment of this aspect and all other aspects described herein, the method further comprises combination therapy with another chemotherapeutic agent.

In another embodiment of this aspect and all other aspects described herein, the inhibitor targets a regulator of mitosis or chromosomal segregation.

In another embodiment of this aspect and all other aspects described herein, the regulator of mitosis or chromosomal segregation is a gene product selected from the group consisting of cyclin A2 (CCNA2), hMIS18a, hMIS1813, C21ORF45, OIP5, borealin (CDCA89), KNL-1 (CASC5), MCAK (KIF2C), subunits of the APC/C complex (ANAPC1, ANAPC4, CDC16, CDC27), SMC4, and the mitotic kinase PLK-1.

In another embodiment of this aspect and all other aspects described herein, the inhibitor is a small molecule selected from the group consisting of paclitaxel, nocodazole, monastrol, and BI-2536.

In another embodiment of this aspect and all other aspects described herein, the inhibitor targets a regulator of APC/C or proteasomes.

In another embodiment of this aspect and all other aspects described herein, the regulator of APC/C or proteasomes is a gene product selected from the group consisting of APC1/ANAPC1, APC4/ANAPC4, Cdc16, Cdc27, PSMA5, PSMB5, and PSMB6.

In another embodiment of this aspect and all other aspects described herein, the method further comprises activating EMI1, or EV15.

In another embodiment of this aspect and all other aspects described herein, the method further comprises combination treatment of the individual with BI-2536.

In another embodiment of this aspect and all other aspects described herein, the inhibitor is MG132, or bortezomib.

In another embodiment of this aspect and all other aspects described herein, the inhibitor is selected from the antibodies listed in Table 7. Alternatively, in another embodiment of this aspect and all other aspects described herein, the inhibitor is selected from the miRNA listed in Table 8.

Also described herein is a method for determining prognosis in an individual having an activating Ras mutation, the method comprising: (a) measuring the levels of COPS3, Cdc16, and EV15 in a test sample from an individual having an activating Ras mutation; and (b) comparing the levels of COPS3, Cdc16 and EV15 to the levels of COPS3, Cdc16, and EV15 in a reference sample, wherein a decreased level of COPS3, a decreased level of Cdc16 and an increased level of EV15 compared to the reference sample indicates a good prognosis, and wherein a larger degree of change indicates a more positive prognosis.

In one embodiment of this aspect and all other aspects described herein, the test sample is a biopsy sample.

In another embodiment of this aspect and all other aspects described herein, the reference sample is obtained from the same individual.

In another embodiment of this aspect and all other aspects described herein, the reference sample is obtained from the individual prior to onset of a detectable cancer.

In another embodiment of this aspect and all other aspects described herein, the reference sample is obtained from a non-cancerous tissue.

In another embodiment of this aspect and all other aspects described herein, the reference sample is obtained from a population of individuals.

Also described herein is a method for inhibiting growth or survival of a cell bearing an activating Ras mutation, the method comprising contacting a cancer cell with an inhibitor of at least one gene from Table 2, wherein the inhibitor inhibits growth or survival of the cancer cell.

In one embodiment of this aspect and all other aspects described herein, the gene is selected from the group consisting of ACVR1c, ANAPC4, BUB1, CAND1, CLDN1, COPS3, F8, FABP3, FBXO3, HGS, JAK1, KCNA3, LMTK3, MAP4K4, MAPK1, MYST3, PI4K2B, PKN1, PLK1, PPP1R10, PRKCB1, PTPRE, RAD51C, RAF1, RNF20, SENP1, SENP8, SP100, TDRD3, TERF1, TRIM54, UCKL1, and XPO1.

In another embodiment of this aspect and all other aspects described herein, the gene is selected from the group consisting of ANAPC4, COPS3, JAK1, PLK1, and XPO1.

In another embodiment of this aspect and all other aspects described herein, the cancer cell is in culture.

In another embodiment of this aspect and all other aspects described herein, the cancer cell is a colon cancer cell.

In another embodiment of this aspect and all other aspects described herein, the cancer cell is a breast cancer cell.

In another embodiment of this aspect and all other aspects described herein, the inhibitor is selected from the group consisting of an RNA interference molecule, a small molecule, an antibody, an aptamer and a nucleic acid.

In another embodiment of this aspect and all other aspects described herein, the contacting step further comprises treating with an inhibitor of a plurality of genes.

In another embodiment of this aspect and all other aspects described herein, the method further comprises combination therapy with another chemotherapeutic agent.

Also described herein is a method for treating cancer in an individual, the method comprising administering an inhibitor of at least one gene from Table 3, 4 or 5 to an individual having cancer, wherein the inhibitor treats cancer in the individual.

In another embodiment of this aspect and all other aspects described herein, the gene is selected from the group consisting of ACVR1C, ANAPC4, BUB1, CAND1, CLDN1, COPS3, F8, FABP3, FBXO3, HGS, JAK1, KCNA3, LMTK3, MAP4K4, MAPK1, MYST3, PI4K2B, PKN1, PLK1, PPP1R10, PRKCB1, PTPRE, RAD51C, RAF1, RNF20, SENP1, SENP8, SP100, TDRD3, TERF1, TRIM54, UCKL1, and XPO1.

In another embodiment of this aspect and all other aspects described herein, the gene is selected from the group consisting of ANAPC4, COPS3, JAK1, PLK1, and XPO1.

In another embodiment of this aspect and all other aspects described herein, the cancer is colon cancer.

In another embodiment of this aspect and all other aspects described herein, the cancer is breast cancer.

In another embodiment of this aspect and all other aspects described herein, the inhibitor is selected from the group consisting of an RNA interference molecule, a small molecule, an antibody, an aptamer and a nucleic acid.

In another embodiment of this aspect and all other aspects described herein, the administering step comprises administering an inhibitor to a plurality of genes.

In another embodiment of this aspect and all other aspects described herein, the method further comprises combination therapy with a chemotherapeutic agent.

### DEFINITIONS

As used herein, the terms “down-modulation” refers to reducing the function of a target gene. This can be accomplished by directly affecting the gene itself, (e.g., by reducing gene expression or protein synthesis), or alternatively by reducing target function/activity at the protein level. As such, an agent useful in the methods described herein is one that inhibits target gene expression or protein synthesis, or inhibits target protein function or activity.

As used herein, the term “inhibitor of at least one gene” is an agent that selectively “down-modulates” at least one desired target gene. An inhibitor can be any agent capable of down-modulating a target gene including, but not limited to, a small molecule, an antibody, an RNA interference molecule, an aptamer, or a nucleic acid. As used herein, the term “inhibitor” means an agent that down-modulates a measurable indicator of target gene expression and/or protein activity in cells by at least 5% compared to the expression or activity of the target gene in the absence of the agent. Preferably, an inhibitor down-modulates expression and/or activity of a target gene by at least 10%, at least 20%, at least 30%, at least 40%, at least 50%, at least 60%, at least 70%, at least 80%, at least 90%, at least 95%, at least 99%, or even 100% (i.e., absent) compared to expression and/or activity of a target gene in the absence of the agent. In certain aspects, the expression and/or activity of more than one gene is down-modulated, for example, 2 target genes, 3, 4, 5, 6, 7, 8, 9, 10, 20, 40, 50, or more genes.

As used herein, the term “activating Ras mutation” refers to any mutation in the Ras oncogene that results in enhanced activity of the Ras polypeptide as assessed by e.g., activation of one or more downstream pathways of Ras. By “enhanced activity” is meant an increase in Ras activity by at least 5% compared to a reference control. Three Ras genes have been identified in the mammalian genome (designated H-ras, K-ras, and N-ras), which acquire cancer cell transformation-inducing properties by single point mutations within their coding sequences. For example, a commonly detected activating Ras mutation found in human tumors is in codon 12 of the H-ras gene in which a base change from GGC to GTC results in a glycine-to-valine substitution in the GTPase regulatory domain of the Ras protein product. This single amino acid change is thought to abolish normal control of Ras protein function, thereby converting it from a normally regulated cellular protein to one that is constitutively active. This de-regulation of normal Ras protein function permits transformation of a cell from a state of normal growth to a state of malignant growth. Downstream effectors of Ras activation and the Ras pathway are further described by Karnoub and Wienberg et al. (2008) Nature: Molecular Cell Biology 9(7): 517-531, which is herein incorporated by reference in its entirety.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

In one embodiment, the agent is an RNA interference molecule. The term “RNAi” and “RNA interfering” with respect to an agent of the invention, are used interchangeably herein. RNAi molecules are typically comprised of a sequence of nucleic acids or nucleic acid analogs, specific for a target gene. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA).

As used herein an “siRNA” refers to a nucleic acid that forms a double stranded RNA, which double stranded RNA has the ability to reduce or inhibit expression of a gene or target gene when the siRNA is present or expressed in the same cell as the target gene, for example an HDF gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length). An siRNA can be chemically synthesized, it can be produced by in vitro transcription, or it can be produced within a cell specifically utilized for such production.

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi and/or siRNA species but differs in that shRNA species are double stranded hairpin-like structure for increased stability. These shRNAs, as well as other such agents described herein, can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The terms “microRNA” or “miRNA” are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### BRIEF DESCRIPTION OF THE FIGURES

FIG. 1. Overview of the pool-based dropout screen with barcode microarrays.

A. Schematic of library construction and screening protocol.

B. Schematic of the HH barcode hybridization.

C. Comparison between HH Amplicons (top) and full-hairpin PCR amplicons (bottom) on an HH probe microarray.

FIG. 2. Pool-based dropout screen for genes required for cancer cell viability.

A. Overview of shRNA pool behavior in the screen. For each cell line, shRNAs were ranked on the basis of their mean normalized log₂ Cy3/Cy5 ratios. The shaded rectangle indicates the log₂ ratio range within which an shRNA's abundance was considered unchanged.

B. Clustering of the four cell lines with the anti-proliferative shRNAs identified in the screen. The shaded scale represents mean normalized log₂ Cy3/Cy5 ratios of the probes.

C. Anti-proliferative shRNAs and genes that scored in the screen for each cell line are shown.

D. Summary of the common shRNAs and genes identified in the screen. Overlapping anti-proliferative shRNAs/genes between pairwise combinations of cell lines are displayed (DLKD-1 and HMEC have more overlapping genes than shRNAs because, in some cases, different sets of shRNAs targeting the same gene scored in each line).

FIG. 3. Genes commonly required for proliferation or survival of normal and cancer cells.

A. Representative candidate shRNAs that reduce viability of all four cell lines. Multiple entries for the same gene indicate that multiple independent shRNAs have scored in the screen for that gene. For each shRNA, the normalized log₂ Cy3/Cy5 ratio (i.e., its relative abundance in the end sample versus initial sample) is given.

B. Examples of core cellular modules required in all four cell lines. Shown are the APC/C (top left), the COP9 signalosome (top right), the eIF3 translation initiation complex (bottom left), and E3 ubiquitin ligases (bottom right).

C. Validation of selected shRNAs from the screen that reduce viability of all four cell lines. Candidate shRNAs were individually packaged into viruses and infected into cells in 96-well in independent triplicates. Cell viability was measured at day 9 after infection. All viability reductions were significant (P<0.05), except where indicated by the number sign. FF, negative control shRNA targeting firefly luciferase.

FIG. 4. Genes selectively required for proliferation or survival of cancer cells.

A. Identification of PPP1R12A (one shRNA) and PRPS2 (two shRNAs) as two genes that are selectively required by HCC1954 or DLD-1 cells respectively, in the screen.

B. and C. Validation of PPP1R12A (B) and PRPS2 (C) as selectively required for viability of HCC1954 or DLD-1 cells, respectively. Cells were either infected with individual retroviral shRNAs or transfected with individual siRNAs in triplicates. Cell viability was measured at 9 days after infection (shRNA) or 4 days after transfection (siRNA) (*, P<0.05). Luc, negative control siRNA targeting luciferase. PLK1, positive control siRNA targeting polo-like kinase 1.

D. Normalized log₂ ratios of an MDM2 shRNA in the screen.

E. shRNA knockdown of MDM2 selectively impairs the viability of HMECs. Cell viability was measured 9 days after infection with retroviruses expressing five different MDM2 shRNAs (* P<0.05).

F. Differential sensitivity of the four cell lines to the MDM2 inhibitor nutlin-3. Cell viability is reflective of their p53 status (HMECs and HCT116 cells, p53 wild-type; HCC1954 and DLD-1 cells, p53 mutant). Cell viability was measured after 4 days of nutlin-3 treatment (*, P<0.05). ctrl, control.

G. Normalized log₂ ratios of a BUB1 shRNA from the screen.

H. Enhanced sensitivity of HCC1954 cells to BUB1 knockdown. Both shRNA (left) and siRNA (right) knockdown of BUB1 reduce HCC1954 cell viability but have no effect on HMEC viability. Cell viability was measured 4 or 9 days after transfection or infection, respectively (* P<0.05).

FIG. 5. Quality assessment of the half-hairpin hybridization methodology.

A. Genomic DNA from library-infected HCT116 cells was independently amplified by PCR twice to recover the HHs. The two PCR products were labeled with Cy3 and Cy5 dyes, respectively, and hybridized competitively to a HH microarray. The raw Cy3 and Cy5 signals were plotted for each probe.

B. Cy5 signal intensities of microarray spots for HH PCR products for 3 separate pools of infected cells. Negative control probes are in gray, experimental HH probes are in black. Probes were ranked based on their mean signal intensity and error bars represent standard deviations across triplicates. The line indicates the cutoff at 2-fold above the mean signal of background probes.

C. HH PCR products from sub-pool 1.1 plasmids (4,223 shRNAs) were hybridized to a microarray containing probes to sub-pools 1.1, 1.2 and 1.3 (12,852 probes). The percentage of probes in the indicated range of signal intensity for sub-pools 1.1 (specific hybridization) and sub-pool 1.3 (cross-hybridization) are shown.

D. Genomic DNA from cells infected with three sub-pools (1.1, 1.2 and 1.3) of shRNA were mixed at different ratios (1:3:8 or 1:1:1) and equal amounts of total DNA were PCR amplified, labeled separately with Cy3 and Cy5, respectively, and hybridized to a single microarray containing HH probes against all 3 sub-pools. A scatter plot is displayed with each sub-pool in a different shade to illustrate the separation of signals. The input DNA ratio for each sub-pool is indicated.

E. Quantification of data in D, with probes in each sub-pool binned by signal intensity and plotted for mean log₂ Cy3/Cy5 ratio. Error bars indicate standard deviations.

FIG. 6. Verification of protein knockdown by shRNAs and siRNAs.

A. Western blots of RBX1 protein knockdown in the 4 human cell lines by RBX1 shRNAs (4 days post-infection) as indicated in FIG. 3C. Protein levels were quantified by first normalizing to loading control (tubulin) and then calculated as a percentage of negative control shRNA (FF, a hairpin targeting firefly luciferase).

B. Western blots of PPP1R12A protein knockdown in DLD-1 and HCC1954 cells by PPP1R12A shRNAs (4 days post-infection) and siRNAs (3 days post-transfection) as indicated in FIG. 4B (Luc, siRNA duplex targeting luciferase).

C. Western blots of PRPS2 protein knockdown in DLD-1 and HCC1954 cells by PRPS2 shRNAs (4 days post-infection) as indicated in FIG. 4C.

D. Top, Western blots of MDM2 protein knockdown in HCC1954 cells and HMECs by MDM2 shRNAs (4 days post-infection) as indicated in FIG. 4E. Bottom, MDM2 and p21 induction in response to nutlin-3 (2 days post treatment) as indicated in FIG. 4F.

E. Western blots of BUB1 protein knockdown in HCC1954 cells and HMECs by BUB1 shRNAs and siRNAs as indicated in FIG. 4H.

FIG. 7. A large-scale pool-based shRNA screen in mouse ES cells.

A. Mean log₂ Cy3/Cy5 ratios for all informative probes in the screen.

B. Representative candidate shRNAs that reduce ES cell viability. For each shRNA, the normalized log₂ ratio is given. Multiple entries for the same gene name indicate multiple independent shRNAs have scored in the screen for that gene. Error bars represent standard deviation across triplicates.

C. Functional classification (based on GO category) of the identified candidate proliferation genes for ES cells by either biological processes (left) or molecular functions (right).

FIG. 8. Scheme of the Ras synthetic lethal screen.

A. Schematic of the primary screen. Change in a particular shRNA's abundance in the pool over time is tracked by competitive hybridization between the initial sample (PD 0) and the final sample (PD 17). A low Cy3/Cy5 ratio indicates the dropout of an anti-proliferative or cytotoxic shRNA from the pool. Synthetic lethal shRNAs are identified as those that selectively drop out from the Ras Mut cells but not Ras WT cells. PD, population doubling; HH, half-hairpin.

B. MAPK pathway activity in DLD-1 cells. Phosphorylation on p42/p44 Erk kinases in cells that were either in full media, serum starved overnight, or serum-starved and then stimulated with full media.

C. Growth curve of DLD-1 cells in culture.

D. Anchorage independence growth of DLD-1 cells in soft-agarose. Colony formation was assessed 2 weeks after seeding.

E. Schematic of the competition assay used to validate shRNAs from the primary screen. Ras Mut cells expressing GFP and Ras WT cells were mixed and co-infected with the same retroviral shRNA. The Mut to WT cell ratio at the end of the experiment is measured by FACS. The percentage of Ras Mut cells in the mixture infected with a candidate RSL shRNA was normalized against that of a control shRNA targeting firefly luciferase (FF) within the same experiment to generate a normalized fitness score for the Ras Mut cells. Variations of this assay are also used to test the effect of siRNAs and chemical inhibitors.

FIG. 9. Functional diversity of candidate RSL genes.

A. Functional classification of candidate RSL genes based on biological processes as annotated in PANTHER. The P-value denotes selective enrichment for hits in the particular biological process.

B. Validation of candidate RSL genes with multiple shRNAs using the competition assay. FF, negative control shRNA targeting firefly luciferase (for each shRNA p<0.01 compares to the respective FF control, except * p<0.05). Error bars indicate standard deviations in all figures unless otherwise indicated.

FIG. 10. Hypersensitivity of Ras Mut cells to mitotic stress.

A. Examples of mitotic genes with multiple shRNAs showing synthetic lethality with mutant Ras (for each shRNA p<0.01 compares to respective FF control, except * p<0.05 and # not significant).

B. Mitotic index in asynchronous DLD-1 cells growing in log-phase (* p<0.05).

C. Ras Mut DLD-1 cells show higher frequency of abnormal anaphase as measured by cells with lagging chromosome 40 minutes after released from metaphase block by the Eg5 kinesin inhibitor monastrol (* p<0.05). An example of Ras Mut cell in anaphase with lagging chromosome (arrowhead) is shown.

D. The microtubule stabilizer paclitaxel selectively decreases the fitness of Ras Mut cells in a dose dependent fashion. The competition assay was carried out in the presence of paclitaxel for 5 days (* p<0.05,** p<0.01 compared to untreated samples).

E. Paclitaxel preferentially induces the G2 and mitotic accumulation of Ras Mut DLD-1 cells as assessed by FACS using DNA and phospho-H3 Ser10 staining, respectively (** p<0.01).

F. Paclitaxel causes strong prometaphase arrest in mitotic DLD-1 Ra Mut cells (shown are mean values of independent triplicates).

FIG. 11. Hypersensitivity of Ras Mut cells to PLK1 inhibition.

A. PLK1 knockdown by shRNA leads to enhanced toxicity in Ras Mut cells (for each shRNA p<0.01 compares to respective FF control, except * p<0.05).

B. The PLK1 inhibitor BI-2536 selectively decreases the fitness of Ras Mut cells in a dose-dependent fashion. The competition assay was carried out in the presence of BI-2536 for 5 days (* p<0.05,** p<0.01 compared to untreated samples).

C. Effect of synthetic lethal concentrations of BI-2536 on cell cycle distribution of Ras Mut and Ras WT DLD-1 cells after 26 hours of treatment. Cell cycle profiles shown are representative of 3 experiments.

D. BI-2536 causes strong prometaphase arrest in mitotic DLD-1 Ras Mut cells (shown are mean values of independent triplicates).

E. BI-2536 does not differentially affect mitotic entry in DLD-1 Ras Mut and WT cells. Cells synchronized at the G2/M boundary by the CDK1 inhibitor RO-3306 were released into nocodazole (100 ng/ml) together with indicated concentrations of BI-2536 for 1 hour. Mitotic index was measured as the percentage of cells staining positive for phospho-H3 Ser10.

F. BI-2536 differentially affect mitotic progression in DLD-1 Ras Mut and WT cells. Mitotic cells collected by nocodazole shake-off were released into indicated concentrations of BI-2536 for 2 hours. Mitotic index was measured as the percentage of cells staining positive for phospho-H3 Ser 10 (* p<0.05,** p<0.01 compared to samples without BI-2536 treatment).

G. Effect of BI-2536 (25 nM) on cell cycle distribution of DLD-1 Ras Mut and WT cells over a 48-hour period (* p<0.05, ** p<0.01).

FIG. 12. Ras Mut cells are hypersensitive to APC/C and proteasome inhibition.

A. Multiple shRNAs against the APC/C subunits APC1 and APC4 confer synthetic lethality in Ras Mut cells as measured by the competition assay (for each shRNA p<0.05 compares to respective FF control, except # not significant).

B. Over expression of the APC/C inhibitor EMI1 and its binding protein EVI5 selectively impair the viability of Ras Mut cells days (* p<0.05 compared to uninfected samples).

C. siRNA mediated knockdown of APC/C subunits synergizes with low concentration of BI-2536 to selectively impaired the viability of DLD-1 Ras Mut cells as measured by the competition assay. Cells were transfected with pools of 4 siRNAs against each gene, 2 days post transfection cells were treated with 10 nM of BI-2536 for 3 days before analysis by FACS (* p<0.05 compared to untransfected samples in the sample treatment group).

D. shRNAs targeting various proteasome subunits exhibit synthetic lethality in Ras Mut cells as measured by the competition assay (for each shRNA p<0.05 compares to respective FF control, except # not significant).

E. The proteasome inhibitors MG132 and bortezomib selectively decreased the fitness of DLD-1 Ras Mut cells in a dose dependent fashion. The competition assay was carried out in the presence of the drug for 4 days (* p<0.05,**p<0.01 compared to untreated samples).

F. MG132 and bortezomib preferentially induced the accumulation of Ras Mut DLD-1 cells as assessed by FACS using staining (** p<0.01 compared to samples without drug treatment).

G. BI-MG132 and bortezomib cause strong prometaphase arrest in mitotic DLD-1 Ras Mut cells (shown are mean values of independent triplicates).

FIG. 13. A potential model of mitotic regulation by Ras.

Without wishing to be bound by theory, the findings presented herein support a model that oncogenic Ras introduces mitotic stress by interfering with kinetochore and APC/C function.

FIG. 14. Candidate RSL genes associated with prognosis in human lung adenocarcinomas showing activation of the Ras pathway.

A. A gene expression signature for lung cancers with activated Ras pathway. A previously-derived gene expression signature (766 genes) of KRAS mutant versus wild-type lung tumors was used as a probe in an additional set of expression profiles from 442 human lung adenomcarcinomas. Both tumors with significant (P<0.01) similarity and tumors with dissimilarity to the KRAS signature (“Ras signature +” and “Ras signature −” tumors, respectively) were considered for subsequent survival analyses.

B. RSL pathway genes that correlate with prognosis among “Ras signature+” tumors. The expression of two candidate RSL genes, COPS3 and CDC16, are inversely correlated with better survival whereas the expression of EVI5 correlates with better survival (P<0.05). Within each of the two tumor subsets considered (“Ras signature +” and “Ras signature −”), tumors with expression levels for the given gene greater than the median were compared to the rest of the tumors in the subset, using Kaplan-Meier analysis.

C. Prognosis in “Ras signature +” and “Ras signature −” tumors using combined information from COPS3, CDC16, and EVI5. For each of the three genes, “+” and “−” is relative to their median expression across the subset of tumors. Within each of the two tumor subsets, three tumor groups were compared by Kaplan-Meier analysis: tumors with high CDC16, high COPS3, and low EVI5 (“CDC16+ COPS3+EVI−”); tumors with low CDC16, low COPS3, and high EVI5 (“CDC16−COPS3−EVI+”); and tumors not falling into these two groups (“other”). Log-rank P-values indicate significant differences among any of the three groups.

FIG. 15. Effect of K-Ras knockdown in DLD-1 and HCT116 cells.

A. DLD-1 Ras Mut cell fitness is modestly decreased by K-Ras shRNAs as measured by the competition assay (**p<0.01 compares to respective FF control).

B. Effect of K-Ras shRNAs on cell proliferation, colony formation on adherent surface and colony formation in soft agarose in DLD-1 and HCT116 Ras Mut cells. For the proliferation assay, equal number of cells stably expressing either K-Ras shRNAs or a control firefly luciferase (FF) shRNA were seeded at normal density and cell numbers were estimated 4 days later using ClelTiter-GLO assay. For adherent colony formation, cells were seeded at low density on adherent surface and colonies were counted 10 days later. For anchorage independent colony formation, cells were seeded at low density in soft agarose media and colonies were counted 3 week later (p<0.01 for all shRNAs compares to respective FF control).

C. Representative images of the colony assays in B for K-Ras shRNA1 in DLD-1 cells.

D. K-Ras protein knockdown in cell lines stably expressing K-Ras shRNAs were verified by Western blotting. Numbers under the blot indicate normalized levels of K-Ras proteins after adjusting for loading.

E. Effect of the MEK inhibitor U0126 and the PI3K inhibitor LY294002 on the fitness of DLD-1 Ras Mut cells in the competition assay. The assay was carried out for 5 days in the presence of various drug concentrations as indicated.

FIG. 16. Knockdown of COPS4 impair DLD-1 Ras Mut cell viability.

A. Effect of COPS4 shRNAs on cell proliferation, colony formation on adherent surface and colony formation in soft agarose in DLD-1 Ras Mut cells (p<0.05 for all shRNAs compared to respective FF control, except # not significant).

B. Representative images of the colony assays in B for K-Ras shRNA1 in DLD-1 cells.

C. Knockdown efficiency of COPS4 shRNAs in DLD-1 cells.

FIG. 17. Sensitivity of Ras Mut cells to nocodazole and paclitaxel.

A. Nocodazole shows no synthetic lethality with mutant Ras. The relative fitness of DLD-1 and HCT116 Ras Mut cells at various concentrations of Nocodazole was measured by the competition assay.

B. DLD-1 Ras Mut cells are hypersensitive to paclitaxel-induced cell cycle arrest.

FIG. 18. Hypersensitivity of Ras Mut cells to PLK1 inhibition.

A. PLK1 knockdown by siRNA leads to enhanced toxicity in DLD-1 Ras Mut cells. Luc, negative control siRNA targeting firefly luciferase (p<0.01 for all siRNAs compared to respective Luc siRNA control).

B. BI-2536 shows enhanced toxicity in DLD-1 Ras Mut cells as measured by CellTiter GLO cell viability assay (** p<0.01).

C. Representative image of DLD-1 cells treated with 50 nM BI-2536 for 24 hours. Mitotic cells at various stages were identified based on phospho-H3 Ser10 (pH3S10) staining, chromosome morphology (DAPI staining) and mitotic spindle arrangement (tubulin staining). Arrowhead indicates cell with a metaphase plate on bipolar spindle. Whereas a significant number of metaphase Ras WT cells still show fully aligned chromosomes under this condition, few metaphase Ras Mut cells are found to have fully aligned chromosomes.

D. BI-2536 does not differentially affect mitotic entry in DLD-1 Ras Mut and WT cells. Cell synchronization and release scheme is shown on top. Cells synchronized at the G2/M boundary by the CDK1 inhibitor RO-3306 were released into nocodazole (100 ng/ml) together with indicated concentrations of BI-2536 for 1 hour. Mitotic index was measured as the percentage of cells staining positive for phospho-H3 Ser10 (pH3S10).

E. BI-2536 differentially affects mitotic progression in DLD-1 Ras Mut and WT cells. Cell synchronization and release scheme is shown on top. Mitotic cells collected by nocodazole shake-off were released into indicated concentrations of BI-2536 for 2 hours. Mitotic index was measured as the percentage of cells staining positive for phospho-H3 Ser10.

F. Effect of BI-2536 (25 nM) on cell cycle distribution of Ras Mut and Ras WT DLD-1 cells over a 48-hour period.

G. PLK1 protein level and activation (as assessed by T210 phosphorylation) in Ras Mut and WT DLD-1 cells. Numbers below each blot indicates normalized signal intensity after an adjustment for loading. Asyn, asynchronous population; S (TT), S phase population arrested by double thymidine block; M (Noc) and M (BI), M phase population collected by releasing from double thymidine block into nocodazole (200 ng/ml) and BI-2536 (100 nM), respectively; G2/M (RO), G2/M population collected by releasing from double thymidine block into the CDK1 inhibitor RO-3306 (10 uM). Each inhibitor is used at concentrations that cause complete cell cycle arrest as verified by FACS.

FIG. 19. Hypersensitivity of HCT116 Ras Mut cells to proteasome inhibition.

A. The proteasome inhibitors MG132 and bortezomib selectively decrease the fitness of HCT116 Ras Mut cells. The competition assay was carried out in the presence of drug for 4 days (p<0.01 compared to untreated samples).

B. MG132 and bortezomib preferentially induce the accumulation of Ras Mut HCT116 cells as assessed by FACS using staining (p<0.05 compared to untreated samples).

FIG. 20. Synthetic lethal effect of mitotic inhibitors when applied as a transient treatment.

The effect of transient treatment with BI-2536, paclitaxel, bortezomib and MG132 on the fitness of DLD-1 Ras Mut cells was measured using competition assay. Cells were treated for a 24 hour period with drugs at the indicated concentrations. Drugs were then washed out and cells were allowed to proliferate for 3 additional days before FACS analysis.

FIG. 21. Lack of synthetic lethality of various mitotic inhibitors with the PI3K oncogene.

A. The effect of BI-2536, paclitaxel, bortezomib and MG132 on the fitness of DLD-1 PI3K Mut cells vs. PI3K WT cells as measured by the competition assay.

B. The effect of BI-2536, paclitaxel, bortezomib and MG132 on the fitness of HCT116 PI3K Mut cells vs. PI3K WT cells as measured by the competition assay.

Table 1. Candidate Ras synthetic lethal shRNAs and genes

Table 2. Validated Ras synthetic lethal shRNA and genes

Table 3. shRNAs that selectively impaired the viability of DLD-1 cells and not HMECs

Table 4. shRNAs that selectively impaired the viability of HCT116 cells and not HMECs

Table 5. shRNAs that selectively impaired the viability of HCC1954 cells and not HMECs

Table 6. Short list of exemplary gene targets

Table 7. Exemplary antibody inhibitors and exemplary epitope sequences

Table 8. Exemplary miRNA sequences

Table 9. Sequence information of shRNAs used in the screen.

### DETAILED DESCRIPTION OF THE INVENTION

The present invention relates to the treatment of cancer, for example, breast and colon cancer. A method based on RNA-interference was developed and experiments were carried out to identify cellular genes whose function is required for cancer cells but not normal cells. The protein products of these genes represent ideal drug targets for the treatment of cancer because inhibitors against these proteins would selectively impair the viability of cancer cells but not that of normal cells.

The target genes or gene products described herein serve as effective targets for treatment of cancer. In one embodiment, the method involves down-modulating one or more target genes or inhibiting one or more target gene products. Down-modulation can be achieved by contacting a cell with an agent that down-modulates the target gene. The agent can be formulated to enhance specific uptake or delivery to the interior of the cell as required.

### Ras Oncogene

The Ras family of small GTPases are frequently mutated in human cancers and are among the most studied oncogenes (Karnoub and Weinberg, 2008). Ras is a membrane-bound signaling molecule that cycles between the inactive, GDP-bound state and the active, GTP-bound state. Growth factor receptor signaling promotes GTP loading and activation of Ras, which in turn activates an array of downstream pathways to promote cell proliferation and survival. Among the major Ras effector pathways are the MAP kinase pathway (Zhang et al., 1993; Warne et al., 1993; Vojtek et al., 1993; Moodie et al., 1993), the PI 3-kinase (PI3K) pathway (Rodriguez-Viciana et al., 1994; Rodriguez-Viciana et al., 1997), RalGDS proteins (Spaargaren and Bischoff, 1994; Kikuchi et al., 1994; Hofer et al., 1994; Chien and White, 2003), phospholipase-Cε (Kelley et al., 2001; Song et al., 2001; Lopez et al., 2001) and Rac (Kelley et al., 2001; Song et al., 2001; Lopez et al., 2001), each of these has been implicated in mediating the tumorigenic effect of the Ras oncogene. Ras GAPs (GTPase activating proteins) inactivate Ras by stimulating its GTP hydrolysis (Bernards and Settleman, 2004). Oncogenic mutations in Ras are invariably point mutations that either interfere with Ras GAP binding to Ras or directly disrupts Ras GTPase activity, and therefore lock Ras in a constitutively active, GTP-bound state. Oncogenic mutations have been found in all three members of the Ras gene family, KRAS, HRAS and NRAS, with KRAS being the most frequently mutated member. KRAS mutations are found at high frequencies in pancreatic, thyroid, colon, lung, liver cancers and in myelodyspastic syndrome (Karnoub and Weinberg, 2008) and are correlated with poor prognosis (Andreyev et al., 2001; Mascaux et al., 2005).

Oncogenic H-, K-, and N-Ras arise from point mutations limited to a small number of sites (amino acids 12, 13, 59 and 61). Unlike normal Ras, oncogenic ras proteins lack intrinsic GTPase activity and hence remain constitutively activated (Trahey, M., and McCormick, F. (1987) Science 238: 542-5; Tabin, C. J, et al, (1982) Nature, 300: 143-9; Taparowsky, E. et al. (1982) Nature. 300; 762-5). The participation of oncogenic ras in human cancers is estimated to be 30% (Almoguera, C. et al (1988) Cell. 53:549-54),

Mutations are frequently limited to only one of the ras genes, and the frequency is tissue- and tumor type-specific. K-ras is the most commonly mutated oncogene in human cancers, especially the codon-12 mutation. While oncogenic activation of H-, K-, and N-Ras arising from single nucleotide substitutions has been observed in 30% of human cancers (Bos, J. L. (1989) Cancer Res 49, 4682-9), over 90% of human pancreatic cancer manifest the codon 12 K-ras mutation (Almoguera, C. et al, (1988) Cell 53, 549-54; Smit, V. T, et al. (1988) Nucleic Acids Res 16, 7773-82; Bos, J. L., (1989) Cancer Res 49, 4682-9). Pancreatic ductal adenocarcinoma, the most common cancer of the pancreas, has a rapid onset and is often resistant to treatment. The high frequency of K-ras mutations in human pancreatic tumors indicates that constitutive Ras activation plays a critical role during pancreatic oncogenesis. Adenocarcinoma of the exocrine pancreas represents the fourth-leading cause of cancer-related mortality in Western countries, Treatment has had limited success and the five-year survival remains less than 5% with a mean survival of 4 months for patients with surgically unresectable tumors (Jernal, A et al (2002) CA Cancer J 52, 23-47; Burris, E1, A. 3rd et. al, (1997) J Clin Oncol 15, 2403-13). This point mutation can be identified early in the course of the disease when normal cuboidal pancreatic ductal epithelium progresses to a flat hyperplastic lesion, and is considered causative in the pathogenesis of pancreatic cancer (Hruban, R. H. et al (2000) Clin Cancer Res 6, 2969-72; Tada, M. et al. (1996) Gastroenterology 110, 227-31).

K-ras mutations are present in 50% of the cancers of colon and lung (Bos, J. L. et al. (1987) Nature. 327: 293-7; Rodenhuis, S. et al. (1988) Cancer Res. 48: 5738-41). In cancers of the urinary tract and bladder, mutations are primarily in the H-ras gene (Fujita, J. et al. (1984) Nature. 309: 464-6; Visvanathan, K. V. et al. (1988) Oncogene Res. 3: 77-86). N-ras gene mutations are present in 30% of leukemia and liver cancer. Approximately 25% of skin lesions in humans involve mutations of the Fla-Ras (25% for squamous cell carcinoma and 28% for melanomas) (Bos, J. L. (1989) Cancer Res. 49:4683-9; Migley, R. S. and Kerr, D. J. (2002) Crit Rev Oncol Hematol. 44:109-20).50-60% of thyroid carcinomas are unique in having mutations in all three genes (Adjei, A. A, (2001) J Nati Cancer Inst. 93: 1062-74).

Constitutive activation of Ras can be achieved through oncogenic mutations or via hyper-activated growth factor receptors such as the EGFRs. Elevated expression and/or amplification of the members of the EGFR family, especially the EGFR and HER2, have been implicated in various forms of human malignancies (as reviewed in Prenzel, N. et al. (2001) Endocr Relat Cancer. 8:11-31). In some of these cancers (including pancreas, colon, bladder, lung), EGFR1HER2 overexpression is compounded by the presence of oncogenic Ras mutations. Abnormal activation of these receptors in tumors can be attributed to overexpression, gene amplification, constitutive activation mutations or autocrine growth factor loops (Voldborg, B. R. et al. (1997) Ann Oncol. 8:1197-206). For growth factor receptors, especially the EGFRs, amplification or/and overexpression of these receptors frequently occur in the cancers of the breast, ovary, stomach, esophagus, pancreatic, lung, colon neuroblastoma.

### Gene Down-Modulation

Gene down-modulation can be achieved by inhibition of protein expression (e.g., transcription, translation, post-translational processing) or protein function. Any composition known to inhibit or down-modulate one or more of the target genes disclosed herein can be used for down-modulation.

An example of a down-modulatory agent of the present invention is gene silencing of the target gene, such as with an RNAi molecule (e.g., siRNA, shRNA, miRNA, etc.). This entails a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the RNAi. In one preferred embodiment, the mRNA levels are decreased by at least about 70%, about 80%, about 90%, about 95%, about 99%, about 100%.

Another aspect of the invention relates to the agent that down-modulates the target gene, and formulations and compositions in which it is contained. Any known inhibitor or down-modulator of the target identified herein can be used as a down-modulating agent in the present methods. In addition, new agents are identified herein as useful as a down-modulatory agent in the treatment of cancer in a subject.

### Agents

Agents useful in the methods as disclosed herein may inhibit gene expression (i.e. suppress and/or repress the expression of a gene of interest (e.g., the target gene)). Such agents are referred to in the art as “gene silencers” and are commonly known to those of ordinary skill in the art. Examples include, but are not limited to a nucleic acid sequence, (e.g., for an RNA, DNA, or nucleic acid analogue). These can be single or double stranded. They can encode a protein of interest, can be an oligonucleotide, a nucleic acid analogue. Included in the term “nucleic acid sequences” are general and/or specific inhibitors. Some known nucleic acid analogs are peptide nucleic acid (PNA), pseudo-complementary PNA (pc-PNA), locked nucleic acids (LNA) and derivatives thereof. Nucleic acid sequence agents can also be nucleic acid sequences encoding proteins that act as transcriptional repressors, antisense molecules, ribozymes, small inhibitory nucleic acid sequences, such as RNAi, shRNAi, siRNA, micro RNAi (miRNA), antisense oligonucleotides. Many of these molecular functions are known in the art. As such these inhibiting can function as an agent in the present invention. In one embodiment, the RNAi comprises the nucleic acid sequences listed in the Table 9 for use in down-modulating the corresponding gene. Additional sequences may also be present. In another embodiment, the RNAi comprises a fragment of at least 5 consecutive nucleic acids of the sequences listed for use in down-modulating the corresponding gene listed.

Such an agent can take the form of any entity which is normally not present or not present at the levels being administered to the cell or organism. Agents such as chemicals; small molecules; nucleic acid sequences; nucleic acid analogues; proteins; peptides; aptamers; antibodies; or fragments thereof, can be identified or generated for use to downmodulate or inhibit.

Agents in the form of a protein and/or peptide or fragment thereof can also be designed to down-modulate a target. Such agents encompass proteins which are normally absent or proteins that are normally endogenously expressed in the host cell. Examples of useful proteins are mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Agents also include antibodies (polyclonal or monoclonal), neutralizing antibodies, or antibody fragments. A list of exemplary antibodies that can be used as inhibitors are included herein in Table 7. Also included in Table 7 are epitopes useful for generating antibodies using methods known to those of skill in the art. It is also contemplated herein that antibodies generated against these epitopes can be administered to an individual for down-modulating a target protein or for treatment of cancer.

Agents can also include peptides, proteins, peptide-mimetics, aptamers, hormones, small molecules, carbohydrates or variants thereof that function to inactivate the nucleic acid and/or protein of the gene products identified herein, and those as yet unidentified. Inhibitory agents can also be a chemical, small molecule, chemical entity, nucleic acid sequences, nucleic acid analogues or protein or polypeptide or analogue or fragment thereof. The agent may function directly in the form in which it is administered. Alternatively, the agent can be modified or utilized intracellularly to produce something which down-modulates a target, such as introduction of a nucleic acid sequence into the cell and its transcription resulting in the production of the nucleic acid and/or protein inhibitor within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The agent may comprise a vector. Many such vectors useful for transferring exogenous genes into target mammalian cells are available. The vectors may be episomal, e.g., plasmids, virus derived vectors such cytomegalovirus, adenovirus, etc., or may be integrated into the target cell genome, through homologous recombination or random integration, e.g., retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

Many viral vectors or virus-associated vectors are known in the art. Such vectors can be used as carriers of a nucleic acid construct into the cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g. EPV and EBV vectors.

The inserted material of the vectors described herein may be operatively linked to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence. In some examples, transcription of an inserted material is under the control of a promoter sequence (or other transcriptional regulatory sequence) which controls the expression of the recombinant gene in a cell- type in which expression is intended. It will also be understood that the inserted material can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene. The promoter sequence may be a “tissue-specific promoter,” which means a nucleic acid sequence that serves as a promoter, i.e., regulates expression of a selected nucleic acid sequence operably linked to the promoter, and which affects expression of the selected nucleic acid sequence in specific cells, preferably in HIV host cells. The term also covers so-called “leaky” promoters, which regulate expression of a selected nucleic acid primarily in one tissue, but cause expression in other tissues as well.

The agent can be an RNA interference molecule. RNA interference agents can be used with the methods described herein, to inhibit the expression and/or activity of a target gene. “RNA interference (RNAi)” is an evolutionarily conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B., J. of Virology 76(18):9225 (2002), herein incorporated by reference in its entirety), thereby inhibiting expression of the target gene. As used herein, “inhibition of target gene expression” includes any decrease in expression or protein activity or level of the target gene or protein encoded by the target gene as compared to a situation wherein no RNA interference has been induced. The decrease can be of at least 20%, 30%, 40%, 50%, 60%, 70%, 80%, 90%, 95% or 99% or more as compared to the expression of a target gene or the activity or level of the protein encoded by a target gene which has not been targeted by an RNA interfering agent. RNA interfering agents contemplated for use with the methods described herein include, but are not limited to, siRNA, shRNA, miRNA, and dsRNAi. The target gene or sequence of the RNA interfering agent can be a cellular gene or genomic sequence.

An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. Preferably, the siRNA is identical in sequence to its target and targets only one sequence. Each of the RNA interfering agents, such as siRNAs, can be screened for potential off-target effects by, for example, expression profiling. Such methods are known to one skilled in the art and are described, for example, in Jackson et al., Nature Biotechnology 6:635-637 (2003), herein incorporated by reference in its entirety. It is well within the ability of one skilled in the art to design and test for siRNAs that are useful for inhibiting expression and/or activity of a target gene. It is important to note that double-stranded siRNA or shRNA molecules that are cleaved by Dicer in the cell can be up to 100 times more ptent than a 21-mer siRNA or shRNA molecule supplied exogenously (Kim, D H., et al (2005) Nature Biotechnology 23(2):222-226). Thus, an RNAi molecule can be designed to be more effective by providing a sequence for Dicer cleavage.

Methods for effective siRNA design for use in vivo can be found in U.S. Pat. No. 7,427,605, which is herein incorporated by reference in its entirety. Commercially available RNA interference molecules that target a desired gene can be obtained from e.g., Santa Cruz Biotechnology Inc. (Santa Cruz, Calif.), Cell Signaling Technologies (Danvers, Mass.), Sigma-Aldrich (St. Louis, Mo.), and Dharmacon Inc. (Lafayette, Colo.), among others. In general, any method of in vivo delivery of a nucleic acid molecule can be adapted for use with an RNAi interference molecule (see e.g., Akhtar S. and Julian R L. (1992) Trends Cell. Biol. 2(5):139-144; WO94/02595, which are incorporated herein by reference in their entirety). However, there are three factors that are important to consider in order to successfully deliver an RNAi molecule in vivo: (a) biological stability of the RNAi molecule, (2) preventing non-specific effects, and (3) accumulation of the RNAi molecule in the target tissue. The non-specific effects of an RNAi molecule can be minimized by local administration by e.g., direct injection into a tumor or topically. Local administration of an RNAi molecule to a treatment site limits the exposure of the e.g., siRNA to systemic tissues and permits a lower dose of the RNAi molecule to be administered. Several studies have shown successful knockdown of gene products when an RNAi molecule is administered locally. For example, intraocular delivery of a VEGF siRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of an siRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering an RNAi molecule systemically for the treatment of a disease, the RNAi molecule can be either be modified or alternatively delivered using a drug delivery system; both methods act to prevent the rapid degradation of the RNAi molecule by endo- and exo-nucleases in vivo.

Modification of the RNAi molecule or the pharmaceutical carrier can also permit targeting of the RNAi molecule to the target tissue and avoid undesirable off-target effects. RNA interference molecules can be modified by chemical conjugation to lipophilic groups such as cholesterol to enhance cellular uptake and prevent degradation. For example, an siRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an RNAi molecule to an aptamer has been shown to inhibit tumor growth and mediate tumor regression in a mouse model of prostate cancer (McNamara, J O., et al (2006) Nat. Biotechnol. 24:1005-1015).

In an alternative embodiment, the RNAi molecules can be delivered using drug delivery systems such as e.g., a nanoparticle, a dendrimer, a polymer, liposomal, or a cationic delivery system. Positively charged cationic delivery systems facilitate binding of an RNA interference molecule (negatively charged) and also enhance interactions at the negatively charged cell membrane to permit efficient uptake of an siRNA by the cell. Cationic lipids, dendrimers, or polymers can either be bound to an RNA interference molecule, or induced to form a vesicle or micelle (see e.g., Kim S H., et al (2008) Journal of Controlled Release 129(2):107-116) that encases an RNAi molecule. The formation of vesicles or micelles further prevents degradation of the RNAi molecule when administered systemically. Methods for making and administering cationic-RNAi complexes are well within the abilities of one skilled in the art (see e.g., Sorensen, D R., et al (2003) J. Mol. Biol. 327:761-766; Verma, U N., et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic administration of RNAi include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an RNAi molecule forms a complex with cyclodextrin for systemic administration. Methods for administration and pharmaceutical compositions of RNAi molecules and cyclodextrins can be found in U.S. Pat. No. 7,427,605, which is herein incorporated by reference in its entirety. Specific methods for administering an RNAi molecule for the inhibition of e.g., an activating Ras mutation can be found in e.g., U.S. Patent Application No. 20080152654, which is herein incorporated by reference in its entirety.

In some embodiments, in order to increase nuclease resistance in an RNAi agent as disclosed herein, one can incorporate non-phosphodiester backbone linkages, as for example methylphosphonate, phosphorothioate or phosphorodithioate linkages or mixtures thereof, into one or more non-RNASE H-activating regions of the RNAi agents. Such non-activating regions may additionally include 2′-substituents and can also include chirally selected backbone linkages in order to increase binding affinity and duplex stability. Other functional groups may also be joined to the oligonucleoside sequence to instill a variety of desirable properties, such as to enhance uptake of the oligonucleoside sequence through cellular membranes, to enhance stability or to enhance the formation of hybrids with the target nucleic acid, or to promote cross-linking with the target (as with a psoralen photo-cross- linking substituent). See, for example, PCT Publication No. WO 92/02532 which is incorporated herein in by reference.

### Pharmaceutical Compositions

In one embodiment, the agent described herein is an active ingredient in a composition comprising a pharmaceutically acceptable carrier. A “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions.

### Screening Test Agents

Various biochemical and molecular biology techniques or assays well known in the art can be employed to practice the methods described herein. Such techniques are described in, e.g., Handbook of Drug Screening, Seethala et al. (eds.), Marcel Dekker (1st ed., 2001); High Throughput Screening Methods and Protocols (Methods in Molecular Biology, 190), Janzen (ed.), Humana Press (1st ed., 2002); Current Protocols in Immunology, Coligan et al. (Ed.), John Wiley & Sons Inc (2002); Sambrook et al., Molecular Cloning: A Laboratory Manual, Cold Spring Harbor Press (3rd ed., 2001); and Brent et al., Current Protocols in Molecular Biology, John Wiley & Sons, Inc. (ringbou ed., 2003). Screens involve a test agent, which is a candidate molecule, to be used in a screen and/or applied in an assay for a desired activity (e.g., down-modulation of a target gene, inhibition of target protein activity, etc.). A “test agent” is screened to identify molecules that down-modulate a target gene (i.e., an inhibitor).

Test agents or compounds that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins. The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecule organic compounds, e.g., chemical compounds with a molecular weight of not more than about 1,000 or not more than about 500. Preferably, high throughput assays are adapted and used to screen for such small molecules. In some methods, combinatorial libraries of small molecule test agents as described above can be readily employed to screen for small molecule compound that inhibit HIV infection. A number of assays are available for such screening, e.g., as described in Schultz (1998) BioorgMed Chem Lett 8:2409-2414; Weller (1997) Mol Divers. 3:61-70; Femandes (1998) Curr Opin Chem Biol 2:597-603; and Sittampalam (1997) Curr Opin Chem Biol 1:384-91.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to a HDF.

Human antibodies against target proteins can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Some non-limiting examples of epitopes useful for generating humanized antibodies are listed herein in Table 7. Humanized antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using an target or its fragment.

Typically, test agents are first screened for ability to down-modulate a biological activity of an target identified herein. A number of assay systems can be employed in this screening step. The screening can utilize an in vitro assay system or a cell-based assay system. In this screening step, test agents can be screened for binding to a target, altering expression level of the target gene, or modulating other biological or molecular activities (e.g., enzymatic activities) of the protein.

### Dosage and Administration

In one aspect, the methods described herein provide a method for inhibiting growth and survival of a cancer cell in a subject. In one embodiment, the subject can be a mammal. In another embodiment, the mammal can be a human, although the approach is effective with respect to all mammals. In one embodiment, the method comprises administering to the subject an effective amount of a pharmaceutical composition comprising an agent that inhibits Ras activity, in a pharmaceutically acceptable carrier.

The dosage range for the agent depends upon the potency, and includes amounts large enough to produce the desired effect, e.g., a reduction in Ras activity as assessed using a competition assay, such as that described herein in Example 2. The dosage should not be so large as to cause unacceptable adverse side effects. Generally, the dosage will vary with the type of agent or inhibitor (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage will range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in e.g., Ras activity, tumor size, tumor volume etc. (see “Efficacy Measurement” below). Such effective amounts can be gauged in clinical trials as well as animal studies for a given inhibitor.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. For the treatment of tumors, the agent can be administered systemically, or alternatively, can be administered directly to the tumor e.g., by intratumor injection or by injection into the tumor's primary blood supply.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, a agent or inhibitor can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent or inhibitor permits the agent attached to accumulate additively at the desired target site. Antibody-based or non- antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

In some embodiments, an inhibitor may be combined with one or more agents such as chemotherapeutic or anti-angiogenic agents, for the treatment of cancer.

In one embodiment, the dose of an agent or inhibitor administered for treatment of a cancer is less than the dose necessary to prevent total mitotic arrest. An appropriate dosage range for in vivo use can be titrated and selected by first determining a dose of agent that completely abolishes mitosis in a particular cell type in culture (i.e., toxic dose). Working below the toxic dose, a therapeutically effective dose can be estimated by assessing e.g., Ras activity at a variety of doses. This dosage range can be further titrated in vivo as deemed necessary by one of skill in the art, while taking into account such factors as family history of disease, prognostic markers, and severity of disease.

### Efficacy Measurement

The efficacy of a given treatment for a cancer or activating Ras mutation can be determined by the skilled clinician. However, a treatment is considered “effective treatment,” as the term is used herein, if any one or all of the signs or symptoms of, as but one example, cancer are altered in a beneficial manner, other clinically accepted symptoms or markers of disease are improved, or even ameliorated, e.g., by at least 10% following treatment with an inhibitor. Efficacy can also be measured by failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the pathogenic growth of cancer cells; or (2) relieving the disease, e.g., causing regression of symptoms, reducing the size of a tumor; and (3) preventing or reducing the likelihood of the development of a neovascular disease, e.g., an ocular neovascular disease).

An effective amount for the treatment of cancer or an activating Ras mutation means that amount which, when administered to a mammal in need thereof, is sufficient to result in effective treatment as that term is defined herein, for that disease. Efficacy of an agent can be determined by assessing physical indicators of, for example cancer, such as e.g., tumor size, tumor volume, tumor growth rate, metastatic phenotype, etc.

For treatment of a subject with an inhibitor of e.g., Ras activation, the in vivo efficacy of an agent can be assessed by e.g., rate of tumor growth, Ras activity in a biopsy sample, tumor volume, inhibition of neovascular growth or assessing a decrease in various markers of angiogenesis as described herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1

A library of short-hairpin RNAs (shRNAs) was previously developed that targets most of the annotated protein-coding genes in the human genome (published in Silva et al. 2004). These shRNAs are embedded in the sequence context of a naturally occurring micro-RNA miR30, each carries a unique 60 nucleotide barcode sequence, and they can be expressed using either retroviral or lentiviral vectors in cells to achieve stable knockdown of target genes through the mechanism of RNAi. Using this library one can systematically interrogate cells for genes whose loss of function (as a result of shRNA-mediated knockdown of their expression) confer a desired phenotype, such as the selective impairment of cancer cell viability.

A sub-pool of the shRNA library shown in Table 9 was screened, that includes shRNAs targeting all human kinases & phosphatases, genes involved in protein ubiquitination/degradation pathways, and genes that are implicated in human cancer. A retroviral vector was used to deliver this pool of shRNAs into 4 cell lines: the colon cancer cell lines DLD-1 and HCT116, the breast cancer cell line HCC1954, and the normal human mammary epithelial cell line HMEC.

To identify genes whose knockdown impairs cell viability, the relative abundance of their corresponding shRNAs was measured at two time points: an initial time point within 72 hours of the retroviral infection of the shRNA, and an end point after the infected cells have been passaged for several weeks.

A new methodology was developed to track the relative abundance of individual shRNAs in a complex pool using microarray hybridization. This method is referred to herein as “Half-hairpin Hybridization”. Using the polymerase chain reaction (PCR), one can recover the anti-sense half of the shRNA sequence (i.e. the half-hairpin) from genomic DNA and, after dye-labeling, hybridize to a microarray containing the corresponding probes of the complementary (sense) sequences, with high fidelity. shRNA half-hairpins were PCR recovered from genomic DNAs of cells at the initial and end time points, labeled with Cy5 and Cy3 dyes, respectively, and competitively hybridized to a microarray with the corresponding probes. The Cy3:Cy5 signal ratio was measured for each probe, which is a direct reflection of the change in the relative abundance of that particular shRNA species in the cell population over time. A negative Cy3:Cy5 ratio, measured on a log₂ scale, indicates the shRNA has been depleted in the population over time, and therefore its target gene is considered to be necessary for growth processes in the cell of interest. Additional statistical criteria was employed to identify genes necessary for growth and survival for each of the aforementioned 4 cell lines. By comparing the list of genes among these cell lines, genes were identified that were preferentially expressed in cancer cell lines (DLD-1, HCT116 or HCC1954) but not in the normal HMEC cell line were identified.

It is important to note that many of the genes preferentially expressed in cancer cells are necessary for growth, survival or development of normal cells, however the dysregulation of these growth pathways and evasion from cell death mediators leads to pathological growth and/or formation of a tumor. By comparing the expression of genes in cancer cells to that of normal cells permits one to identify the growth pathways that are perturbed in a cancer cell but are appropriately regulated in a normally dividing cell. In this manner, a cancer cell can be effectively targeted for treatment with the methods described herein, while having a minimal effect on normally dividing cells.

Also identified were a number of genes whose knockdown by shRNA specifically impaired the viability of DLD-1, HCT116, HCC1954 and HMECs cells. Genes were identified from this dataset that when knocked down specifically impaired the viability of one of the 3 cancer cell lines (DLD-1, HCT116, or HCC1954), but not that of the normal HMECs. These genes are listed herein in Tables 3, 4 and 5. These tables also provide the log₂ Cy3:Cy5 ratio data to indicate cancer-cell selectivity. Because of the selective requirement of some of these genes by cancer cells but not normal cells, inhibition or down-modulation of the gene or their protein products are useful in the treatment of cancer, particularly colon and breast cancers.

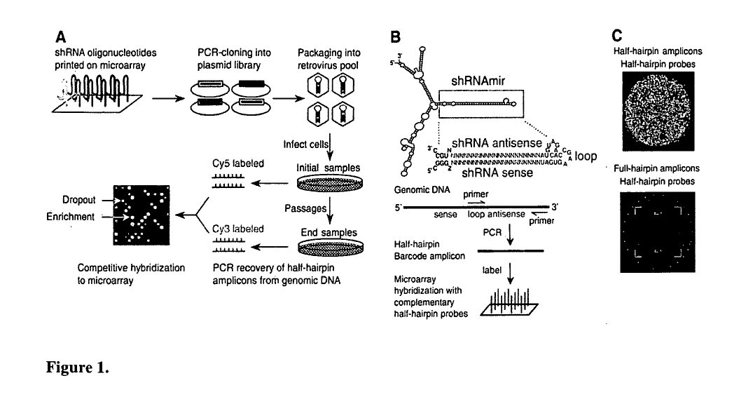

Barcoded, microRNA-based shRNA libraries were generated to target the entire human genome that can be expressed efficiently from retroviral or lentiviral vectors in a variety of cell types for stable gene knockdown (P. J. Paddison et al., Nature 428, 427 (2004); J. M. Silva et al., Nat. Genet. 37, 1281 (2005)). Furthermore, we have also developed a method of screening complex pools of shRNAs using barcodes coupled with microarray deconvolution to take advantage of the highly parallel format, low cost, and flexibility in assay design of this approach (J. M. Silva et al., Nat. Genet. 37, 1281 (2005); T. F. Westbrook et al., Cell 121, 837 (2005)). Although barcodes are not essential for enrichment screens (positive selection) (T. F. Westbrook et al., Cell 121, 837 (2005); N. Popov et al., Nat. Cell Biol. 9, 765 (2007); I. G. Kolfschoten et al., Cell 121, 849 (2005)), they are critical for dropout screens (negative selection) such as those designed to identify cell lethal or drug sensitive shRNAs (V. N. Ngo et al., Nature 441, 106 (2006)). Hairpins that are depleted over time can be identified through the competitive hybridization of barcodes derived from the shRNA population before and after selection to a microarray (FIG. 1A).

The use of 60-mer barcodes for pool deconvolution has been previously described (J. M. Silva et al., Nat. Genet. 37, 1281 (2005); T. F. Westbrook et al., Cell 121, 837 (2005)). To provide an alternative to these bar-codes that enables a more rapid construction and screening of shRNA libraries, methodology called half-hairpin (HH) barcoding for deconvoluting pooled shRNAs was developed. A PCR strategy that amplifies only the 3′-half of the shRNA stem was designed and used from the large 19-nt hairpin loop of our mir30-based platform (FIG. 1B). Compared to using full hairpin sequences for microarray hybridization (K. Berns et al., Nature 428, 431 (2004); T. R. Brummelkamp et al., Nat. Chem. Biol. 2, 202 (2006)). HH barcodes entirely eliminate probe self-annealing during microarray hybridization (FIGS. 1C, 5A and 5B), providing the critical dynamic range necessary for pool-based dropout screens. HH barcode signals are highly reproducible in replicate PCRs (R=0.973, FIG. 5A), highly specific (0.5% cross reaction) (FIG. 5C), and display reasonable, although slightly compressed, dynamic range in mixing experiments where sub-pool inputs are varied in competitive hybridization experiments (FIGS. 5D and 5E). Taken together, these results indicate HH barcodes are alternatives to the 60-mer barcodes originally designed into the library.

A central goal is to develop the means to rapidly perform dropout screens to systematically identify genes required for cancer cell proliferation and survival that could represent new drug targets. The screening platform was used to interrogate human DLD-1 and HCT116 colon cancer cells, human HCC1954 breast cancer cells and normal human mammary epithelial cells (HMECs). Colon and breast cancer cells, two types of cancers with distinct origins, were compared to maximize our ability to identify common and cancer-specific growth regulatory pathways. Recent large-scale efforts have identified a distinct spectrum of mutations in these two cancer types (T. Sjoblom et al., Science 314, 268 (2006); L. D. Wood et al., Science 318, 1108 (2007)). Also, the comparison between cancer and normal mammary epithelial cells can reveal potential growth and survival adaptations specific to cancer cells. A highly complex pool of 8203 distinct shRNAs (Table 9) was constructed to target 2924 genes consisting of annotated kinases, phosphatases, ubiquitination pathway and cancer-related genes (Table 1). These genes were chosen because they are central regulators of signaling pathways that can provide a rich source of phenotypic perturbation. These shRNAs were placed in an MSCV-retroviral vector (R. A. Dickins et al., Nat. Genet. 37, 1289 (2005)), MSCV-PM, that functions efficiently at single copy.

Each cell line was screened in independent triplicates. Cells were infected with an average representation of 1000 per shRNA and a multiplicity of infection (MOI) of 1-2. Initial reference samples were collected 48-72 hours post-infection. The remaining cells were puromycin-selected, propagated for several weeks and collected again as the end samples. HH barcodes were PCR-recovered from genomic DNA, labeled with Cy5 and Cy3 dyes, respectively, and hybridized to a HH barcode microarray (FIG. 1A). The Cy3/Cy5 signal ratio of each probe reports the change in relative abundance of a particular shRNA between the beginning and the end of the experiment. Both correlations between initial samples across the triplicates and between the initial and end samples within each replica were high, indicating the triplicates were highly reproducible and representation was well-maintained throughout the experiment.

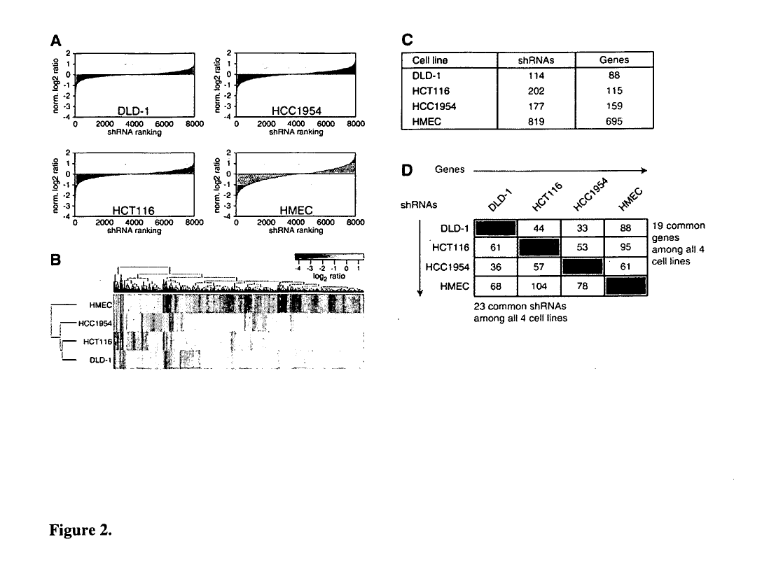

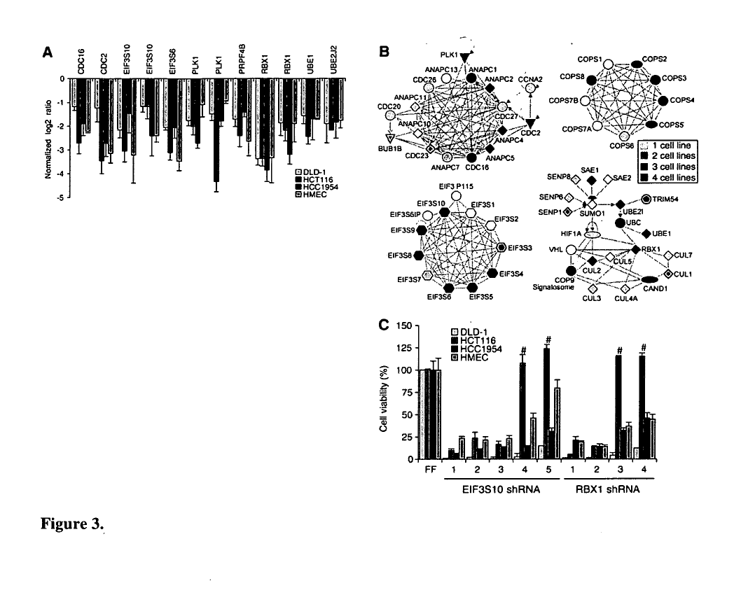

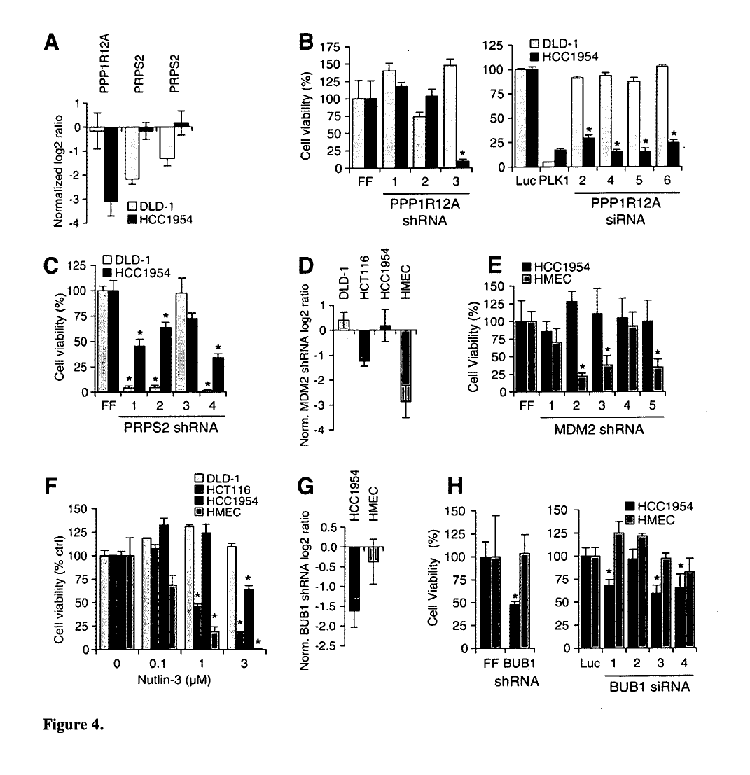

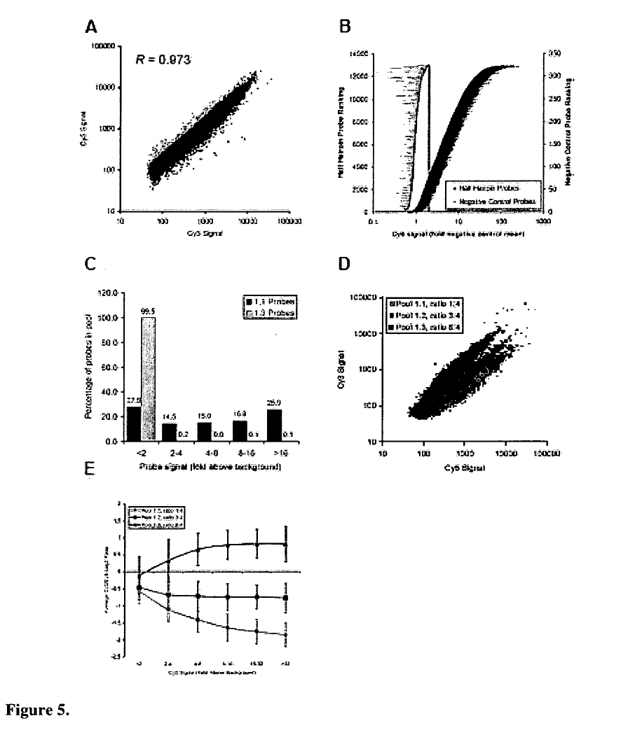

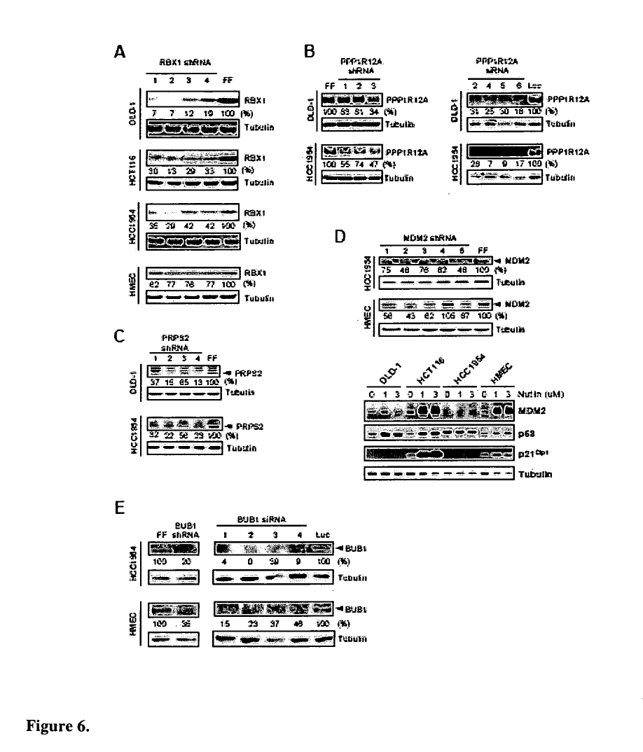

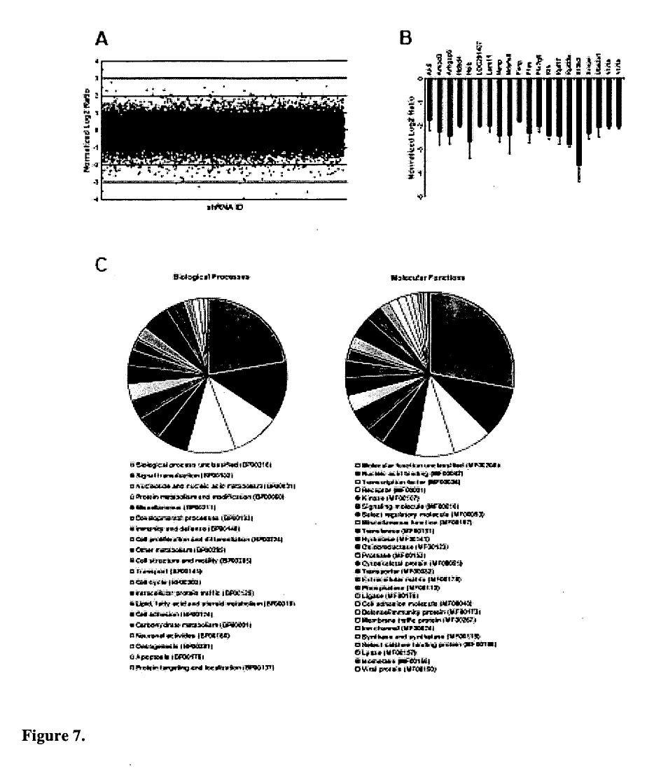

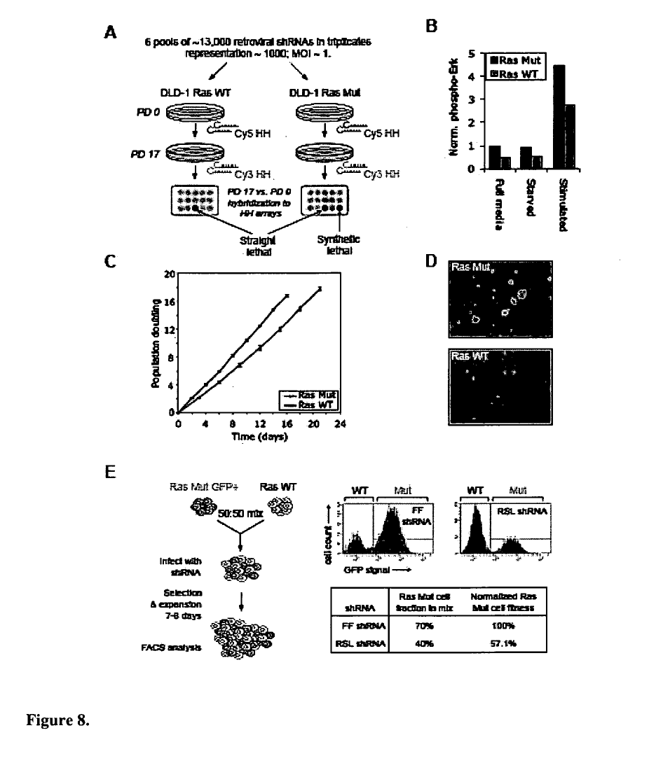

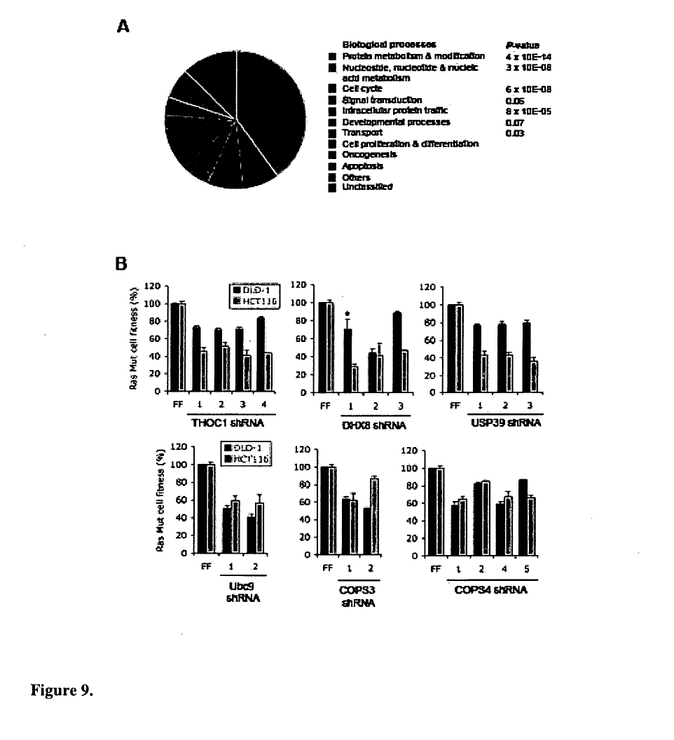

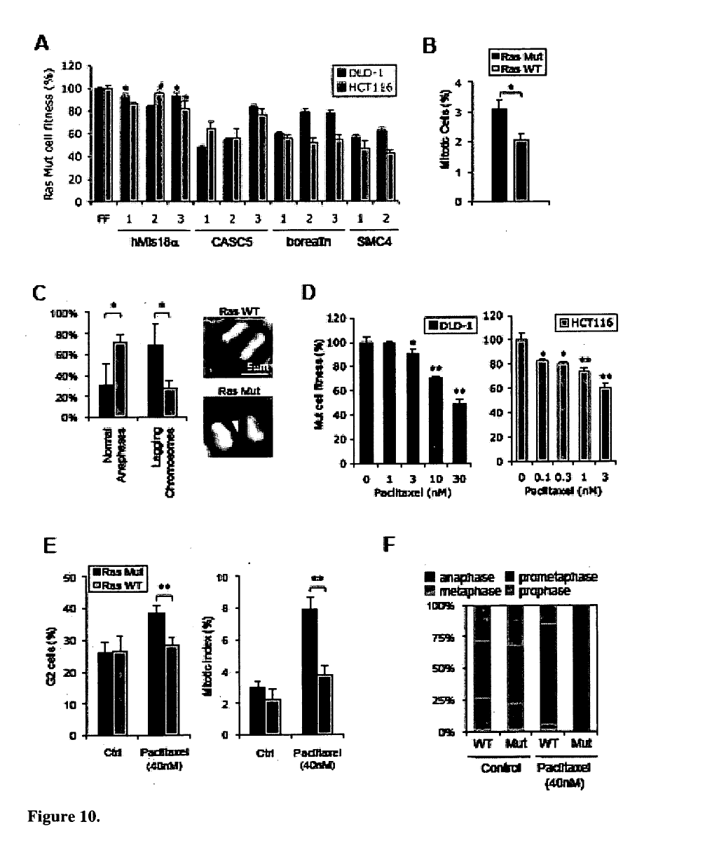

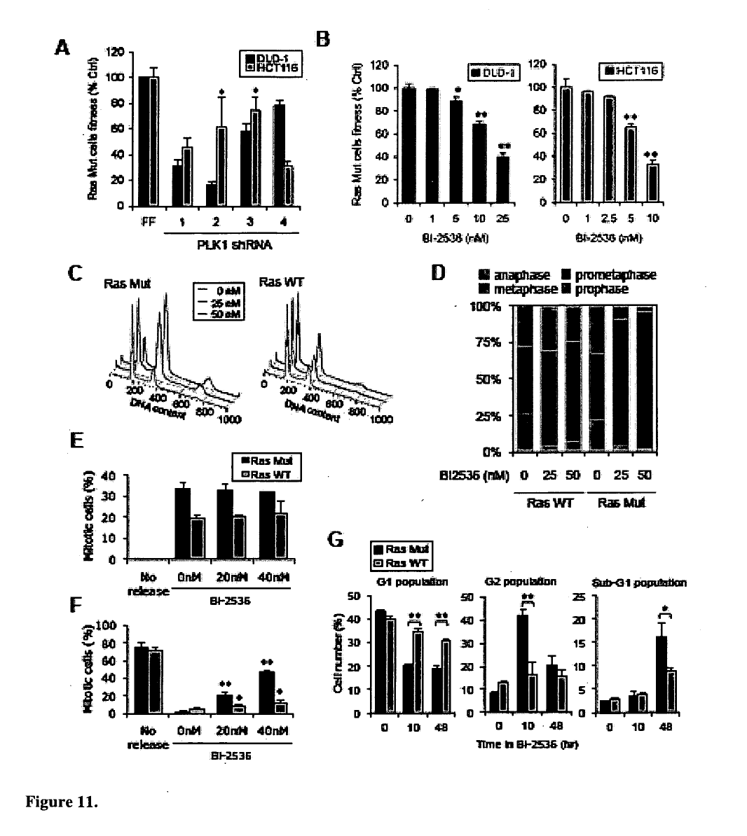

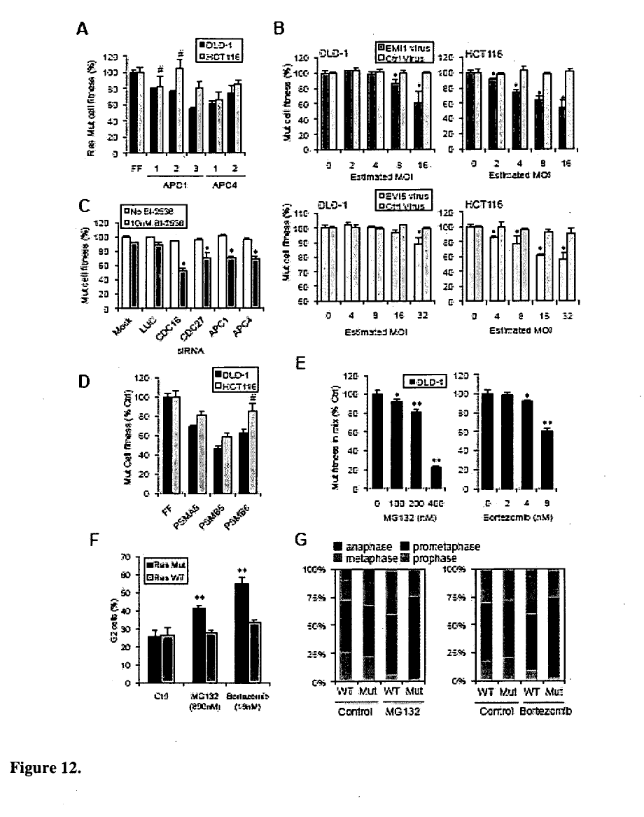

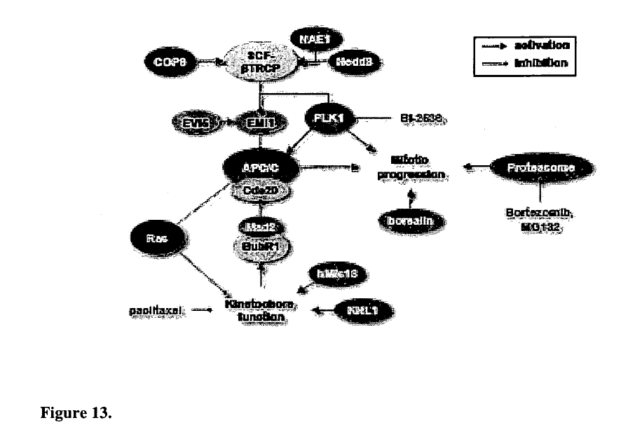

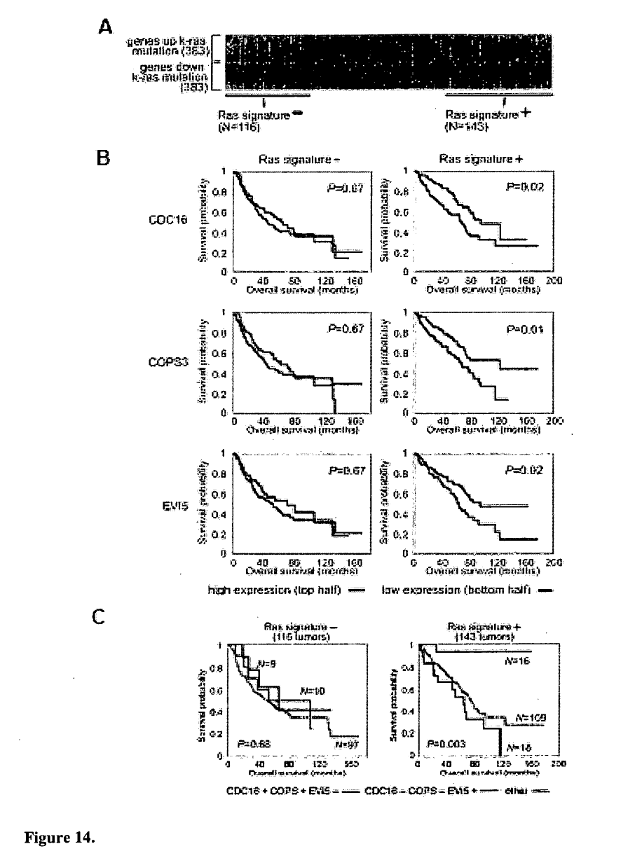

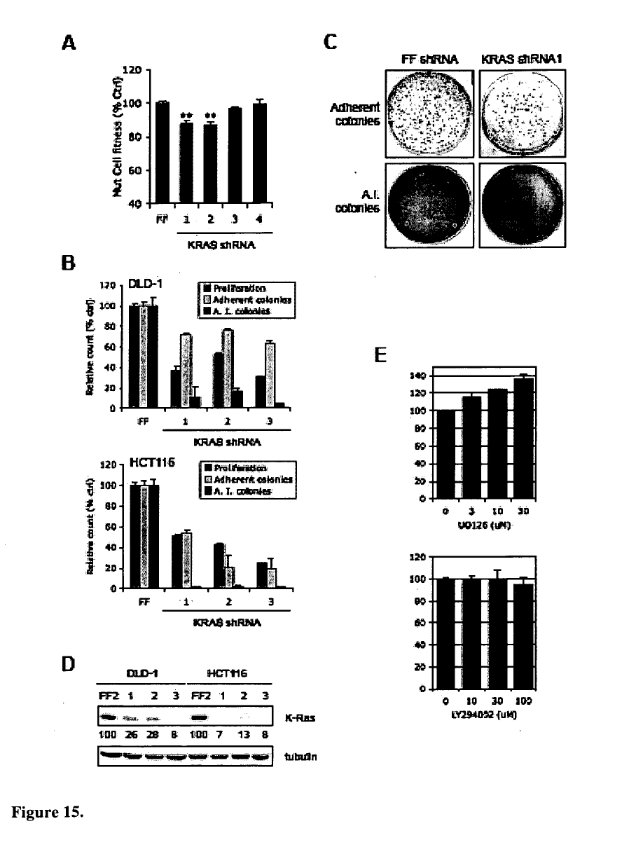

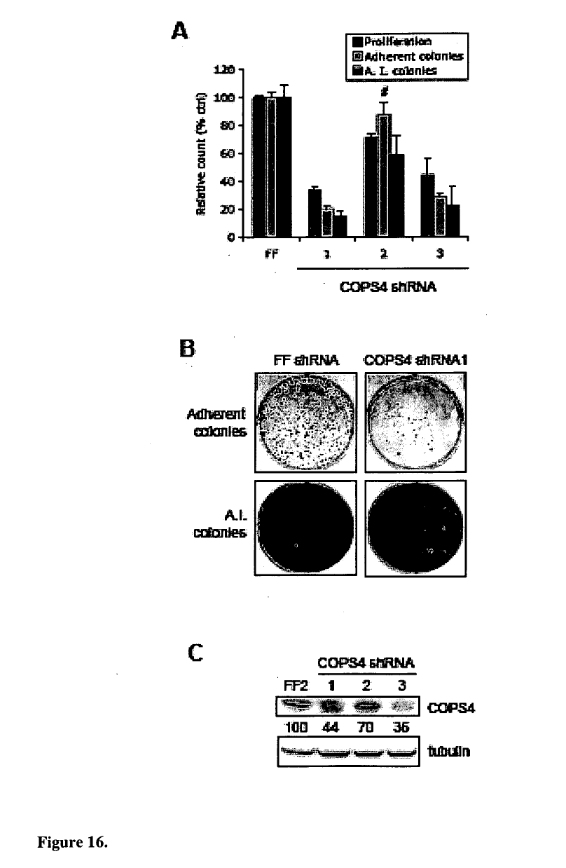

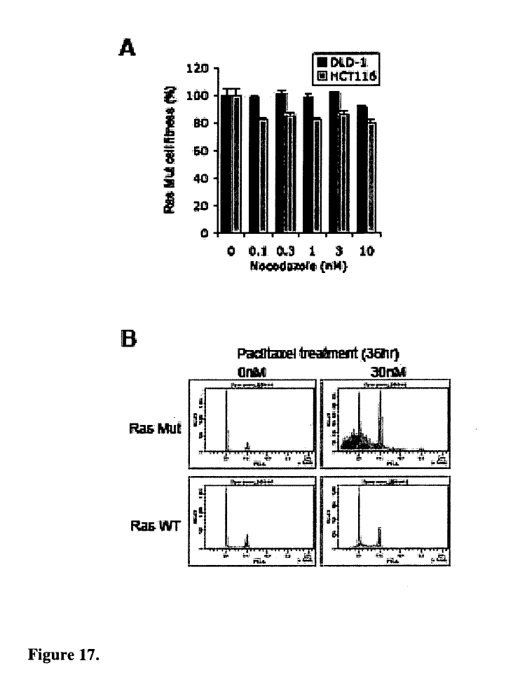

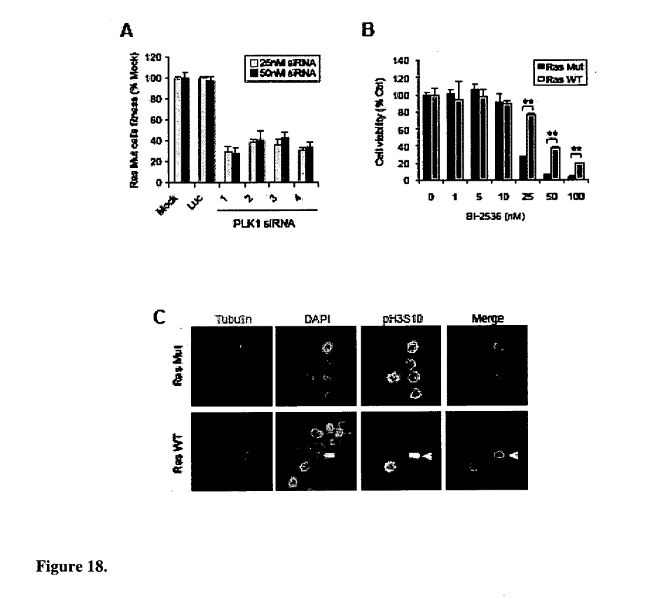

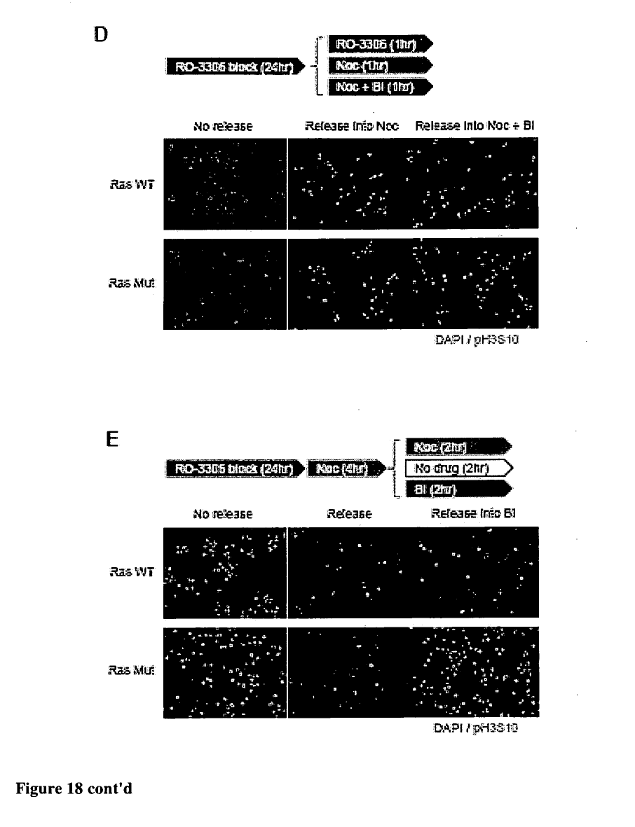

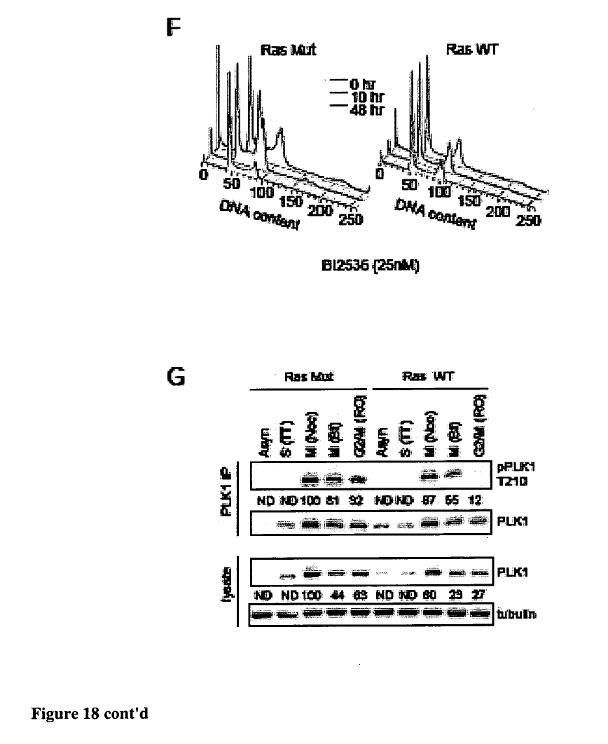

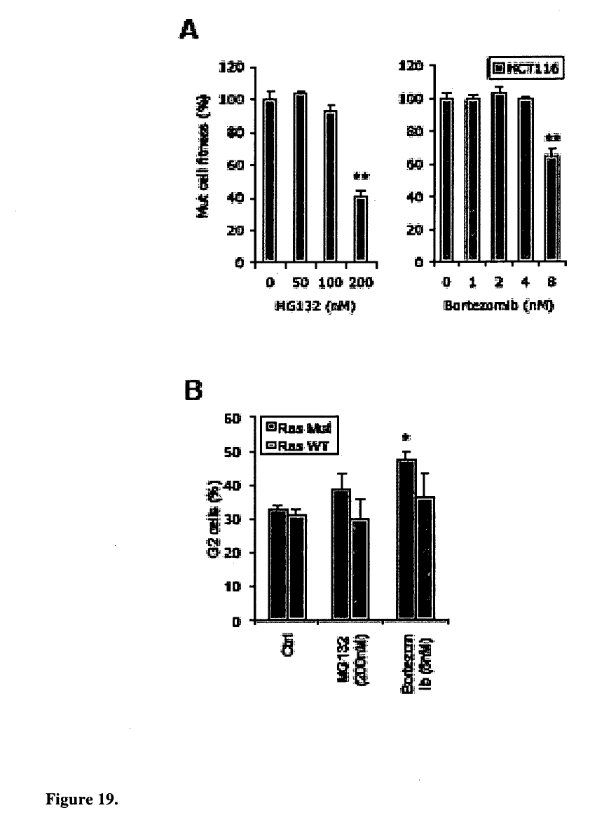

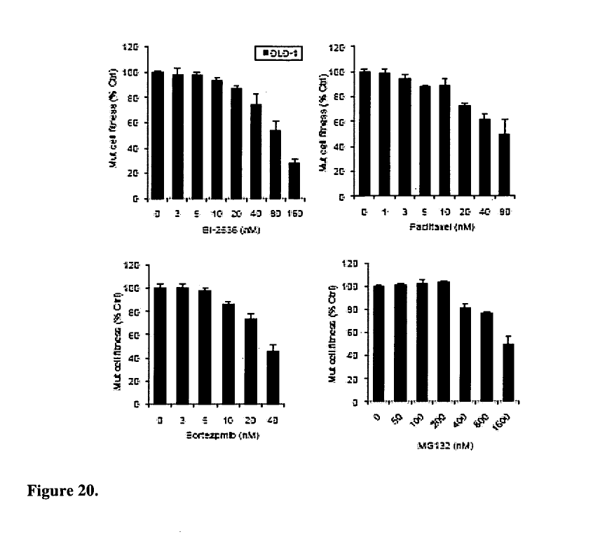

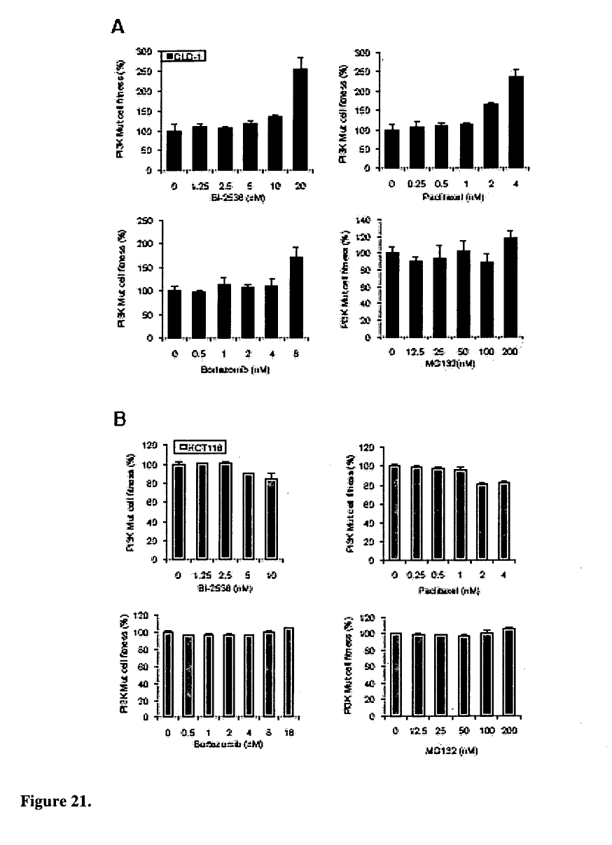

To identify shRNAs that consistently changed abundance in each cell line, datasets were analyzed using a custom statistical package based on the LIMMA method (G. K. Smyth, T. Speed, Methods 31, 265 (2003)) for 2-color cDNA microarray analysis. Whereas the majority of shRNAs show little changes in their abundance over time (log, ratio between −1 and 1), a small fraction of shRNAs showed depletion (FIG. 2A). Based on their shRNA dropout signatures, unsupervised hierarchical clustering segregated the 3 cancer cell lines from the normal HMECs, likely reflecting fundamental differences between cancer and normal cells (FIG. 2B). Furthermore, the two colon cancer cell lines were more similar to each other than the breast cancer line, reflecting the differences in their tissues of origin and paths to tumorigenesis. Overall, 114 shRNAs (1.4%) representing 88 genes (3.0%) in DLD-1 cells were found, 202 shRNAs (2.5%) representing 115 genes (3.9%) were found in HCT116 cells, 177 shRNAs (2.2%) representing 159 genes (5.4%) were found in HCC1954 cells, and 819 shRNAs (10.0%) representing 695 genes (23.8%) were found in HMEC cells to show statistically significant depletion (FIG. 2C). The lists of anti-proliferative shRNAs show significant overlap (p<10⁻⁴⁰), with 23 shRNAs and 19 genes scoring in all four lines (FIG. 2D). As expected, the screen recovered components of core cellular modules essential for all cell lines (FIGS. 3A and 3B). For example, shRNAs against multiple subunits of the anaphase promoting complex (APC/C) (DLD-1 p=9.65×10⁻⁵; HCT116 p=2.99×10⁻⁹; HCC1954 p=1.41×10⁻⁵; HMEC p=5.80×10⁻⁶), the COP9 signalosome (DLD-1 p=2.48×10⁻⁶; HCT116 p=9.34×10⁻⁶; HCC1954 p=4.54×10⁻⁵; HMEC p=0.032), and the eukaryotic translation initiation factor 3 (eIF3) complex (DLD-1 p=1.42×10⁻⁵; HCT116 p=7.98×10⁻⁸; HCC1954 p=0.00024; HMEC p=0.0086) were identified (FIG. 3B). A number of key proteins in the ubiquitination and sumoylation pathways, including most of the cullins, were also identified. Importantly, multiple shRNAs against the same gene were scored in the screen, indicating their effects are unlikely due to off-target effects.

Next EIF3S10 and RBX1, two genes essential for viability in all 4 cell lines were validated. For each gene, shRNAs were included that scored in the screen as well as additional shRNA sequences present in the library (Table 1). Cells were infected with individual retroviral shRNAs and cell viabilities were assessed (FIG. 3C). For each gene, all of the shRNAs that scored in the screen and many additional shRNAs gave anti-proliferative phenotypes. Furthermore, the anti-proliferative activity of the shRNAs correlated very well with the extent of target gene knockdown, as shown for RBX1. Thus, these phenotypes are likely due to target gene knockdown rather than off-target effects. This is consistent with a previous transfection-based screen with this library showing ˜90% “on target” efficiency (V. M. Draviam et al., Nat. Cell Biol. 9, 556 (2007)).

In addition to the common set of shRNAs that impair viability in all cell lines, we observed a substantial number of genes that are selectively required for proliferation of each cell line (Tables 3-5). Without wishing to be bound by theory, changes in these genes may reflect differences in the underlying oncogenic context, and therefore represent potential cancer-selective drug targets. The gene PPP1R12A, which encodes a regulatory subunit of protein phosphatase 1, was validated for its selective requirement in HCC1954 but not DLD-1 cells (FIG. 4A). The PPP1R12A shRNA that gave the greatest depletion (shRNA 3) showed the strongest effect on HCC1954 cells but only marginally affected DLD-1 viability (FIGS. 4B and 6B). This was corroborated with 4 additional PPP1R12A siRNAs. These shRNAs and siRNAs resulted in comparable knockdown of PPP1R12A protein in both cell lines (FIG. 6B), indicating that the selective requirement for PPP1R12A by HCC1954 cells is not due to different degrees of protein knockdown. PPP1R12A has been shown to target protein phosphatase 1 (PP1) isoforms to several substrates including myosin and merlin (M. Ito, T. Nakano, F. Erdodi, D. J. Hartshorne, Mol. Cell. Biochem. 259, 197 (2004); H. Jin, T. Sperka, P. Herrlich, H. Morrison, Nature 442, 576 (2006)). Thus, PP1 activity reduction by PPP1R12A knockdown may lead to increased phosphorylation of key proteins that disrupt the viability of HCC1954 cells. Conversely, PRPS2, which encodes phosphoribosyl pyrophosphate synthetase 2, an enzyme involved in nucleoside metabolism, is more selectively required by DLD-1 than HCC1954 cells (FIGS. 4C and 6C). These results indicate that distinct, genetic context-dependent vulnerabilities exist between these tumor cell lines.

Comparison between HCC1954 cells and normal HMECs also revealed a distinct subset of genes selectively required by each cell line (Table 5). A larger set of 695 genes are required by HMECs, likely reflecting the ability of normal cells to appropriately respond to various cellular stresses. Conversely, the relatively fewer genes required by the cancer cells underscores their ability to evade and overcome growth-inhibitory cues. Among the genes identified as essential for HMECs and HCT116 cells, but not DLD-1 or HCC1954 cells, is HDM2 encoding the human homolog of MDM2, the E3 ligase for p53 (FIG. 4D). HCC1954 and DLD-1 cells harbor inactivating mutations (Y163C and S241F, respectively) in the TP53 gene and are therefore insensitive to MDM2 knockdown. Multiple MDM2 shRNAs selectively impaired the viability of the p53 wildtype HMECs, but not HCC1954 cells with mutant p53 (FIGS. 4E and 6D). Furthermore, this finding was pharmacologically validated by interfering with MDM2 function using the inhibitor nutlin-3 (L. T. Vassilev et al., Science 303, 844 (2004)). and recapitulating the sensitivity of these cells to MDM2 inactivation (FIGS. 4F and 6D).

Importantly, a number of genes appear to be selectively required by HCC1954 cells but not HMECs (Table 5). Among these is the cell cycle regulator and spindle checkpoint kinase BUB1 (FIG. 4G). BUB1 was validated using both shRNA and siRNAs to confirm its knockdown is more detrimental to HCC1954 cells than HMECs (FIG. 4H), despite similar levels of BUB1 protein reduction (FIG. 6E). These results indicate that BUB1 is likely to play an integral role in supporting the oncogenic transformation of HCC1954 cells as they are more dependent on BUB1 function. Without wishing to be bound by theory, one possible explanation for this enhanced dependency may be the near-tetraploid nature of the HCC1954 genome. Compared to the diploid HMECs, HCC9154 cells may rely more heavily on the spindle checkpoint to maintain genomic stability. Such a dependency is an example of “non-oncogene addiction” where cancer cells come to be highly dependent for growth and survival on the functions of genes that are themselves not oncogenes (N. L. Solimini, J. Luo, S. J. Elledge, Cell 130, 986 (2007)).

It is demonstrated herein that highly parallel dropout screens using complex pools of shRNAs can be achieved using HH barcodes in combination with highly-penetrant vectors. The ability to identify anti-proliferative shRNAs specific to particular cell lines indicates that different cancer cells have distinct growth and survival requirements that cluster with cancer type. Targeting such vulnerabilities is one approach for cancer-selective therapeutics. The functional genetic approach demonstrated herein presents an alternative and complementary effort to sequencing-based approaches such as the Cancer Genome Atlas (TCGA) and similar efforts, which focus on physical alterations of the cancer genome.

The most complex pool employed contains 42,000 distinct shRNAs (FIG. 7), an 80-fold increase in complexity compared to previous dropout screens based on these designs (V. N. Ngo et al., Nature 441, 106 (2006)). It is now conceivable to screen the entire human genome with ˜3 shRNAs per gene with a pool of ˜100,000 shRNAs in ˜100 million cells. Thus, a large number of cancer and normal cell lines can be rapidly screened in this manner, with the goal of generating “cancer lethality signatures” for different cancer types and thus identifying cancer type-specific lethal genes representing potential drug targets.

### Methods and Materials

### Cell Culture and Virus Production

HCT116 and DLD-1 colon cancer cells were gifts from Dr. Todd Waldman and Dr. Bert Vogelstein. Both HCT116 cells and DLD-1 cells were maintained in McCoy's 5A media with 10% FBS. HCC1954 breast cancer cells were from American Type Culture Collection (ATCC™) and were maintained in RPMI-1640 media with 10% FBS. HMECs taken from a reduction mammoplasty were immortalized with human telomerase and maintained in MEGM media (LONZAT™). Mouse CCE ES cells were from StemCell Technology, and were maintained in Knockout DMEM (INVITROGEN™) with 15% ES serum (HYCLONE™), 1% non-essential amino acids, 2 mM Glutamine (INVITROGEN™), 0.1 mM b-ME, 1000 U ESGRO (CHEMICON™).

Retroviruses were produced by transfecting 293T cells with MSCV-PM-shRNA, pCG-gag/pol, and pVSV-G plasmids using TranslT-293 (MIRUS®) per manufacturer's instructions. Retroviral supernatant was filtered, titered, and stored at −80° C. until use.

For the screen, HCT116, DLD-1, HCC1954, HMECs, and CCE ES cells were infected using 4-8 μg/ml polybrene (SIGMA®) in 150 mm plates. Three independent infections were carried out for each cell line with an MOI of 1-2 and an average representation of ˜1,000. Viruses were removed 24 h later, and a fraction of cells were collected at 48-72 h post infection as the initial samples (PD=0). Cells were then selected with puromycin (0.5-2 μg/ml) to remove the small number of uninfected cells. Cells were passaged accordingly when reaching ˜80% confluency (except in the case of ES cells, which were passaged every 2 days). For each passage a minimal representation of 1,000 was maintained for the population. For the initial and end samples sufficient cells were collected such that the representation exceeds 1,000. Cell pellets were stored at −80° C.

Vectors, Cloning, and shRNA Libraries

The EcoRI site of pMSCV-puro (CLONTECH®) was converted to an MluI site to generate pMSCV-PM (pMSCV-Puro-MluI). The PheS Gly294 gene was inserted into the XhoI/MluI sites of pMSCV-PM to generate pMSCV-PM-PheS and allow for negative selection on Cl-Phe agar plates when transferring shRNA fragments from pSM2. The second generation Elledge-Hannon human and mouse shRNA libraries were subcloned from the SalI/MluI sites of pSM2c into the XhoI/MluI sites of pMSCV-PM-PheS to generate human and mouse pMSCV-PM-shRNA libraries. A focused set of the human pMSCV-PM-shRNA library consisting of 2813 shRNAs targeting 506 protein kinases, 129 metabolic kinases, and 180 phosphatase catalytic and regulatory subunits, 3549 shRNAs against 837 ubiquitin-proteasome pathway genes (E1-E2-E3 proteins, deubiquitinating enzymes, ubiquitin-binding and ubiquitin-like proteins, proteasome subunits, regulators of cullins, autophagy genes, and components of the EIF3 complex), and 1841 shRNAs targeting 1272 genes implicated in cancer were chosen from the arrayed pSM2c-shRNA library and cloned as pools into the XhoI-MluI sites of pMSCV-PM-PheS as above. All cloning was performed with enzymes from New England Biolabs, Qiaex II or Qiaquick Gel Extraction kits from Qiagen, and DH5α bacteria from Invitrogen as previously described (M. Z. Li, S. J. Elledge, Nat. Genet. 37, 311 (2005)).

### Genomic DNA Preparation, Half-Hairpin Barcode PCR and Probe Labeling

For human cells, genomic DNA was extracted by incubating in 10 mM Tris-HCl pH 8.0, 10 mM EDTA, 0.5% SDS, and 0.2 mg/ml proteinase K, 25 μg/ml RNAse A at 55° C. for 3-12 h, followed by addition of 0.2 M NaCl. For ES cells, genomic DNA was extracted from each replicate of each pool by incubating in 10 mM Tris pH 8.0, 10 mM EDTA, 10 mM NaCl, 0.5% Sarcosyl, 0.1 mg/ml RNAseA at 37° C. for 30 mM and then adding 0.5 mg/ml Proteinase K and incubating at 55° C. for 2 h. Genomic DNA was phenol-chloroform extracted using Phase-Lock tubes (5-Prime), ethanol precipitated, and resuspended in 10 mM Tris-HCl pH 8.0 with 0.1 mM EDTA or 10 mM Tris-HCl pH 8.5.

Pooled genomic DNA was used as template for HH PCR using the forward primer JH353F: TAGTGAAGCCACAGATGTA and one of two reverse primers JH353R: TAATACGACTCACTATAGGGAGTGATTTAATTTATACCATT or BC1R: CCTCCCCTACCCGGTAGA. The 800 μl PCR reaction contained the following final concentrations: 30-60 μg gDNA template, 200 uM dNTPs, 400 nM for each PCR primer, 2% DMSO, 1× Hotstart Taq buffer, and 1 μl Hotstart Taq (TAKARA®). PCR was performed with the following program: 95° C. 5 min, 36 cycles of 94° C. 35 sec, 52° C. 35 sec, 72° C. 1 min, and a final step of 72° C. 10 min. PCR products for each replicate of each time point were pooled, precipitated, resuspended, and gel-purified using a QiaQuick columns (QIAGEN®).

To label the HH PCR products, 2 μg of the PCR product and 2 μg/ml random nonamer primer (final concentration) in 39.25 μl was boiled for 5 min and chilled on ice for 2 min. The reaction was then allowed to proceed at 37° C. for 2 h following the addition of 5 μl 10×NEB buffer 2, 1 μl of 10 mM dATP, dGTP, dTTP, 2.25 μl of Cy3 of Cy5 labeled-dCTP (AMERSHAM®), and 25 U Klenow (NEW ENGLAND BIOLABS™). Reactions were stopped with 5 μl of 0.5 M EDTA pH 8.0 and cleaned using Microcon tubes (YM-30, MILLIPORE™)

### Half-Hairpin Microarray Hybridizations

Custom microarrays of HH probes were synthesized by NIMBELGEN™ at a density of 12×13,000 (12 sub-arrays of 13,000 probes each). Homogeneity of annealing temperature for HH probes was optimized by minor variation in the size of the probes printed on the array through the inclusion of small regions adjacent to the HH to modulate hybridization kinetics and signal intensity. A hybridization mixture for each sub-array containing 180 ng labeled sample of each color, 4 μg/ml CPK6 oligonucleotide, 8 μg/ml Herring sperm DNA, 0.4 mg/ml BSA, and 1×MES Hyb buffer (100 μM MES, 1 M NaCl, 20 mM EDTA, 0.01% Tween-20, 10.5% glycerol) in 10 μl total volume was heated to 95° C. for 90 sec and maintained at 44° C. until loaded on the array. Each sub-array was hybridized with 5.94 μl of the hybridization mixture at 44° C. for 18-19 h. The 12-plex array was washed with 60 ml of stringent wash buffer (SWB, 100 μM MES, 0.1 M NaCl, 0.01% Tween-20) at 44° C. using a syringe through the port on the hybridization chamber followed by a brief wash with room temperature non-stringent wash buffer (NSWB, 6×SSPE 0.01% Tween-20). The array was transferred to warm SWB at 44° C. and incubated for 15 min with gentle agitation every 5 min followed by a brief transfer to NSWB. The array was then incubated in ice-cold 1:100 dilution of NSWB for 30 sec. The array was dried with compressed air and scanned using an Axon 4000B microarray scanner.

Mouse shRNA HH custom arrays were synthesized by Agilent at a density of 4×44,000 (4 sub-arrays of 44,000 probes each). A hybridization mixture for each sub-array consisting of 250 ng Cy5- and 250 ng Cy3-labeled probes, 55 μl 2× Agilent GEX hybridization buffer (Hi-RPM), 11 μl 10× Agilent blocking reagent, and water to a final volume of 110 μl was prepared and 100 μl was added each sub-array. Each sub-array was hybridized at 44° C. overnight, washed as per the manufacturer's wash protocol, and scanned using an Agilent microarray scanner.

### Cell Viability Assay

Cells were infected in triplicate in 96-well or 24-well plates with retroviruses of individual shRNAs as described above, and the media was replaced 24 h after infection. On days 2, 6, or 9 post-infection, cell viability was measured using CellTiter-Glo reagent (Promega) per manufacturer's instruction on a Wallac Victor2 plate reader (Perkin Elmer).

Transfection of siRNAs

Cells were transfected in triplicates using Lipofectamine RNAiMAX reagents (INVITROGEN™) per manufacturer's instructions. The final concentration of siRNAs in the transfection was 50 nM (in the case of PLK1 siRNA SMARTpool, which is a mixture of 4 siRNAs, 50 nM total siRNA) The media was changed after 24 h, and cell viability was assessed 4 days post-transfection as described above.

### Additional Reagents

The following primary and secondary antibodies were used for immunoblotting with the indicated dilutions: rabbit anti-Rbx1 and mouse anti-PRPS2 (1:500, Abcam), rabbit anti-MYPT1 (PPP1R12A gene product, 1:500, Upstate), mouse anti-tubulin and rabbit anti-GAPDH (1:2000, Sigma), mouse anti MDM2 (1:500, Santa Cruz), rabbit anti-BUB1 (1:500, Bethyl), mouse anti-p53 DO-1 and mouse anti-p21 (1:500, Calbiochem), goat anti-rabbit-HRP (1:5000, Jackson ImmunoResearch), and goat anti-mouse-HRP (1:5000, Jackson ImmunoResearch). Positive and negative siRNA controls, including siLUC, siTOX, siGLO duplexes and a PLK1 SMARTpool, were purchased from Dharmacon. Sequences for all shRNAs and siGENOME siRNA duplexes (Dharmacon) tested in the validation experiments are given in Table 9.

### Development of Half-Hairpin Barcode Method

When designing the original barcode strategy for the version 1 shRNA libraries using the U6 promoter and standard short hairpins of 25 bp stem and 6 base loop, the sequence of the hairpin was considered itself as a probe. The problem of probe self-annealing due to the hairpin structure led us to introduce a separate 60-mer barcode sequence into each construct. One potential limitation of this strategy, however, is that it requires extensive sequencing of each new library to generate barcode information and linkage to shRNA sequence. The second generation library design, which places a 21 bp stem and 19 base loop shRNA in the mir30 miRNA backbone, permits employing the shRNA sequences as barcodes for dropout screens using the new, higher-penetrance vectors. PCR primers were designed to anneal to the 19-base loop and the 3′-constant region of the mir30-based shRNA (FIG. 1B). These primers yield a PCR amplicon of 190 bp containing only the 3′-half of the shRNA, herein termed a half-hairpin (HH). Using HH barcodes for microarray hybridization entirely eliminated the problem of probe self-annealing (FIG. 1C), therefore enabling one to carry out pool-based dropout screens.

To assess the efficacy of HH probes, a plasmid DNA pool containing 12,852 unique shRNA hairpins was packaged as a retrovirus and infected into HCT116 human colon cancer cells. Two days after infection, genomic DNA was extracted from the infected cells. To test the reproducibility of the PCR, genomic DNA from a single sample was used as a template in two independent PCR reactions. The two PCR products were labeled with Cy3 or Cy5 dyes and competitively hybridized on a custom microarray containing HH probes for all 12,852 shRNAs. Most probes are 24 nt in length, however, probes with lower melting temperatures (Tₘ) were extended using sequence in the mir30 backbone to obtain a more uniform Tₘ. The Cy5 and Cy3 signal intensities had a correlation coefficient (R) for the pool reaching 0.973 (FIG. 5A) indicating the PCR and labeling reactions were highly reproducible. In addition, less than 3% of the probes exhibited a Cy3/Cy5 ratio greater than 2-fold in either direction, further supporting the reproducibility of the PCR and labeling reactions. Furthermore, independent triplicate infections of the library pools gave highly reproducible hybridization (FIG. 5B).

The utility of HH barcodes was compared with that of full-hairpin barcodes. When the full-hairpin amplicons were hybridized to a microarray containing the corresponding HH probes, they yielded very low signals, with less than 0.8% of the probes reaching >2-fold above background (FIG. 1C, bottom). In comparison, the HH amplicons yielded much stronger hybridization signal in the same experiment (FIG. 1C top and 5B).

The specificity of HH hybridization was tested by examining cross-hybridization of HH amplicons. The test pool of 12,852 plasmid shRNAs was divided into three sub-pools 1.1, 1.2 and 1.3 of approximately equal size (4,200 shRNAs each). Probes derived from sub-pool 1.1, containing 4,223 shRNAs, were hybridized to a microarray containing 12,852 HH probes against the entire pool (FIG. 5C). Of the probes corresponding to shRNAs present in sub-pool 1.1, 72.2% gave a signal >2-fold above background while sub-pool 1.3 probes on the microarray, for which no corresponding DNA was present in the PCR reaction, only 0.5% gave a signal >2-fold above background. Similar results were observed when the other sub-pools were employed as probes. Thus, hybridization of HH probes is highly specific and cross-hybridization occurs infrequently. It was also found through reconstitution mixing experiments of sub-pools that the HH microarrays faithfully reproduced the relative abundance of the different pools (FIGS. 5D and 5E), although the ratios were compressed for probes of low signal strength.

### Screen for Anti-Proliferative Genes in Cancer and Normal Cell Lines

Each screen was carried out in independent triplicates. Cells were infected with the retroviral shRNA pool at an average representation of 1000 per shRNA and an MOI of 1-2. Initial reference samples were collected 48-72 hours post-infection. The remaining cells were puromycin-selected and propagated for several weeks with an average shRNA representation of ≧1000 maintained at each passage. After ˜16 population doublings (PDs) of DLD-1 and HCT116 cells, ˜11 PDs of HCC1954 cells and ˜10 PDs of HMECs, cells were collected again as the end samples. For each replica, HH barcodes were PCR-amplified from genomic DNAs and probes were prepared from the initial and end samples and labeled with Cy5 and Cy3 dyes, respectively. The labeled PCR products were competitively hybridized to a microarray containing the corresponding probe sequences. Most probes (87.2% for DLD-1, 82.5% for HCT116, 84.0% for HCC1954 and 85.9% for HMECs) consistently yielded signal intensities >2-fold above the mean background signals of negative control probes. When 4 standard deviations above the background median was used as the cutoff criterion, 75-80% of probes still consistently made the cutoff.

### Large-Scale Anti-Proliferative Screen in Mouse ES Cells

To test if the screening strategy can be scaled to extremely complex libraries, the most complex library pool available, which was a mouse library consisting of 42,590 shRNAs against 22,996 mouse genes, was used. This library was screened for genes that impaired the viability of mouse embryonic stem (ES) cells. The screen was carried in independent triplicates similar to the protocol described above, and collected the initial samples at day 2 post infection and the end samples after 14 days of further cell passage.

From the microarray, 31,416 (73.8%) of the total 42,590 shRNAs consistently showed hybridization signals >2-fold above background in all 3 replicas in both the initial and the end samples, indicating that the complexity of this very large library was well maintained throughout the screen. More importantly, correlations between the replicate experiments at each time point were very high (0.95±0.02 for day 2 and 0.93±0.01 for day 16 samples), confirming the reliability and effectiveness of the screen. Within the 31,416 shRNAs measured, their log₂ Cy3/Cy5 ratios (FIG. 7A) identified 1116 shRNAs against 1077 genes with a consistent decrease in abundance of >2-fold by SAM with 5% FDR. Examples of candidate shRNAs are shown in FIG. 7B. A large fraction of these ES cell proliferation genes belong to the categories of metabolic enzymes and signal transduction pathway components involved in essential cellular functions (FIG. 7C). Thus, this screening methodology can be applied to very complex libraries with highly reproducible results.

### Statistical Analyses

The microarray datasets were analyzed using a custom statistical package that is based on the LIMMA method for the analysis of 2-color cDNA microarrays. To identify shRNAs that negatively impacted cell viability, intensity-dependent loess normalization was applied to obtain normalized log₂ Cy3/Cy5 ratios for the probes within each replica. This ratio represents the changes in an shRNA's relative abundance between the initial and end samples, with a negative ratio indicating depletion and a positive ratio enrichment over time. Probes with a mean signal <2-fold background from the analysis were removed. Lastly, the method of significance analysis for microarrays (SAM) was applied to identify shRNAs that are consistently depleted across the triplicates. A false-discovery rate (FDR) of 20% as well as Cy3/Cy5 ratio 2-fold cutoff was applied to the dataset for each cell line to identify shRNAs which reduce cell viability in the screen. Interaction network analysis was carried out using Ingenuity Pathway Analysis software (Ingenuity Systems). p values for genes scoring in interaction modules were calculated using a hypergeometric distribution. All p values in FIGS. 3 and 4 were generated using a one-tailed t-test assuming unequal variance. Hierarchical clustering analysis and heatmap generation was conducted using R.

### Example 2

### Treatment of Cells Bearing an Activating Ras Mutation(s)

A major challenge in cancer therapeutics is the identification of cellular drug targets whose inhibition leads to the selective killing of cancer cells while sparing normal cells. Recent advances in mammalian RNA interference (RNAi) technologies have made it possible to systematically interrogate the human genome for genes whose loss of function constitute synthetic lethality either with the oncogenic state or with particular oncogenic mutations (Ngo et al., 2006; Schlabach et al., 2008; Silva et al., 2008). Herein is described barcoded, retroviral/lentiviral-based short hairpin RNA (shRNA) libraries targeting the entire human genome to enable genome-wide loss-of-function analysis through stable gene knockdown (Silva et al., 2005). The design features of this shRNA library permits development of a multiplex screening platform that enables the highly parallel screening of >10,000 shRNAs in a pool-based format using microarray deconvolution for their effect on cell viability (Schlabach et al., 2008; Silva et al., 2008). These technological breakthroughs therefore make it possible to rapidly interrogate the genome for functional vulnerability of cancer cells. This approach is applied in a screen to identify shRNAs that constitute synthetic lethality with the Ras oncogene.

Despite its prominent status as a cancer drug target, therapeutics aimed at disrupting the Ras pathway have proven challenging thus far. Inhibitors of farnysyl transferase, the enzyme that prenylates Ras for its membrane localization, have met with only limited success (Karnoub and Weinberg, 2008). Chemical genetic screens using small molecule libraries in isogenic Ras mutant and wild type cell lines have identified compounds that exhibit preferential toxicity towards Ras muant cells (Bernards and Settleman, 2004; Dolma et al., 2003; Yang and Stockwell, 2008). Inhibitors targeting various Ras effector pathways could also prove efficacious in treat tumors with Ras mutations, as it was recently shown that a combined application of MEK and PI3K/mTOR inhibitors can reduce tumor burden in a mouse model of Ras-driven lung cancer (Engelman et al., 2008).

The systematic identification of genes and pathways necessary for the Ras-driven oncogenic state provides additional drug targets for therapeutic exploration, sheds new light on Ras's mechanisms of action and provide new biomarkers for patient stratification. To this end, the shRNA library was screened for genes whose knockdown constitute synthetic lethality with the KRAS oncogene using isogenic cell lines that are either mutant or wild type for K-Ras. A functionally diverse set of genes were identified that are more critically required for the viability of Ras mutant cells compared to Ras wild type cells, thus revealing a broad genetic networks not previously implicated in supporting the function of the Ras oncogene. In particular, it was discovered that Ras mutant cells are hypersensitive to the depletion of a number of mitotic proteins and demonstrate that pharmacological inhibitors targeting mitotic proteins can selectively impair the viability of Ras mutant cells. These findings point to a previously unappreciated role of Ras in mitotic progression and demonstrate that mitotic stress induced by the Ras oncogene might be exploited for therapeutic purposes.

### Results

### Genome-Wide RNAi Synthetic Lethal Screen Against the KRAS Oncogene

The colorectal cancer cell line DLD-1 was chosen for the primary screen (FIG. 8A). These cells carry a naturally occurring K-Ras G13D point mutation that is required for maintaining the oncogenic state of these cells (Shirasawa et al., 1993; Torrance et al., 2001). An isogenic clone of DLD-1 cells with the KRASG13D allele disrupted shows decreased MAP kinase signaling, reduced proliferation on adherent surface and is no longer able to sustain anchorage independent growth in vitro (FIG. 8B-D) and tumor growth in vivo (Shirasawa et al., 1993). Thus, despite other mutations present in DLD-1 cells, they clearly exhibit addiction to the KRAS oncogene and their malignant phenotype critically depends on mutant K-Ras function.

The parental KRAS WT/G13D (Ras Mut) DLD-1 cells and the isogenic KRAS WT/− (Ras WT) DLD-1 control cells were screened with a library of 74,905 retroviral shRNAs targeting 32,293 unique human gene transcripts (including 19,542 RefSeqs). The library was screened in 6 pools of ˜13,000 shRNAs per pool using a protocol as described previously (FIG. 8A) (Schlabach et al., 2008). For each pool, the change in relative abundance of every shRNA over time was analyzed by microarray hybridization to identify those that are anti-proliferative or cytotoxic and therefore dropout from the population. The lethality signature of the Ras Mut and WT cells were compared to identify those shRNAs that show selective depletion in the Ras Mut cells but not in the Ras WT cells. Such shRNAs are potential Ras synthetic lethal (RSL) candidates. Relaxed statistical criteria identified 1,741 RSL shRNAs targeting 1,613 genes, whereas a more stringent cutoff identified a subset 379 RSL shRNAs targeting 368 genes.

Next, a competition assay was devised to test the reproducibility of candidate RSL shRNAs from the primary screen (Torrance et al., 2001; Smogorzewska et al., 2007). Individual shRNAs were packaged into retroviruses and infected into a 50:50 mixture of GFP+ DLD-1 Ras Mut cells and GF\tilde{P} (colorless) DLD-1 Ras WT cells. The relative ratio of Ras Mut versus WT cells at ˜7 days post shRNA infection was analyzed by FACS and compared to that of a mixed population infected with a negative control shRNA targeting luciferase (FIG. 8E). Using this assay 320 candidate RSL shRNAs from the primary screen were tested and 89 shRNA (27.8%) targeting 82 genes were found to preferentially decrease the fitness of Ras Mut cells compared to Ras WT cells (Table 2). This validation rate is lower than what was observed with “straight lethal” shRNA analysis within individual cell lines (Schlabach et al., 2008). Without wishing to be bound by theory, a possible cause for the higher false positive rate in the primary screen might due to the fact that for the synthetic lethal analysis the difference between two microarray datasets is extracted and coupled with the fact that the secondary assay is much shorter than the original discovery assay. To rule out cell-line specific effect of candidate RSL shRNAs, candidates were tested using the competition assay in a second isogenic pair of colorectal cancer cell lines: HCT116 KRAS WT/G13D and HCT116 KRAS WT/−, which were derived with a similar method as the DLD-1 isogenic pair (Shirasawa et al., 1993; Torrance et al., 2001). Many shRNAs that scored in the DLD-1 cells also showed synthetic lethality in the HCT116 cells (53 of 72 tested, 73.6%, Table 4), indicating the majority of candidate RSL shRNAs are likely to interact genetically with the KRAS oncogene.

### Functional Diversity of Candidate RSL Genes

shRNAs against KRAS itself were recovered from the screen. In accordance with the phenotype of the Ras WT isogenic controls, shRNA-mediated knockdown of K-Ras expression (both Mut and WT protein) in both DLD-1 and HCT116 cells resulted only in modest decrease in growth on adhesive surface but severely impaired colony formation in soft-agarose (FIG. 15), thus confirming the inhibition of K-Ras itself to be sufficient for suppressing the malignant phenotype of these cells. Both the MAP kinase and PI 3-kinase pathways have been implicated in Ras-driven oncogenesis, but a few genes were recovered in these pathways from the primary screen. The effect of the MEK inhibitor U0126 and the PI3K inhibitor LY294002 was tested in these cells. At no concentration of these drugs was selective toxicity towards Ras Mut cells compared to Ras WT cells observed, indicating that inhibiting MEK or PI3K alone in these cells are not sufficient to constitute synthetic lethality (FIG. 15) (Haigis et al., 2008).

The list of candidate RSL genes is functionally diverse (FIG. 9A and Table 9). Using PANTHER (Thomas et al., 2003), several biological processes were identified including protein modification, nucleic acid metabolism, cell cycle and signal transduction that are enriched in the screen. Also identified were a number of molecular pathways that upon which Ras Mut cells are more dependent. For example, shRNAs against genes in ribosomal biogenesis and protein translation control (BXDC2, FBL, NOL5A, EIF3S8, EIF3S4, GSPT1, HNRNPC and METAP1), in protein neddylation (COPS3, COPS4, COPS8, NEDD8, NAE1/APPBP1) and sumoylation pathways (SAE1, UBA2 and UBE2I) and in RNA splicing (FIP1L1, NXF1, USP39, DHX8 and THOC1) were observed. Many of these are growth and survival genes, but reducing their activity leads to enhanced growth defects in Ras Mut cells. To rule out off-target effects, multiple shRNAs from library were tested against several of these candidate genes, and in the majority of the cases one or more additional shRNAs that give the same phenotype were identified (FIG. 9B).

These findings suggest that the Ras oncogene requires additional support from many genes to maintain the oncogenic state. For example, RNA processing/export factor THOC1, a member of the conserved TREX mRNA transport complex, was identified to be synthetically lethal with Ras (FIG. 9B). Indeed, THOC1 has been recently shown to be selectively required for the Ras-driven proliferation and transformation of murine fibroblasts (Li et al., 2007). Also identified were several subunits of the COP9 signalosome [Deshaies Review 2003], which regulates the activity of SCF ubiquitin ligases, whose depletion is synthetically lethal with Ras. Depleting either COPS3 or COPS4 impaired the fitness of Ras Mut cells relative to that of Ras WT cells in the competition assay (FIG. 9). Furthermore, DLD-1 Ras Mut cells with stable depletion of COPS4 exhibit both impaired growth on adherent surfaces and in soft agarose (FIG. 9).

### Ras Mutant Cells are Sensitive to Mitotic Perturbations

A number of genes were identified that are involved in the regulation of mitosis and chromosomal segregation as candidate RSL genes. Among these are cyclin A2 (CCNA2), the hMis18α and hMis18α (C21ORF45 and OIP5), borealin (CDCA8), KNL-1 (CASC5), MCAK (KIF2C), subunits of the APC/C complex (ANAPC1, ANAPC4, CDC16 and CDC27), SMC4 and the mitotic kinase PLK1 (Tables 1 and 2). Their depletion phenotypes are likely to be on-target effects as multiple shRNAs gave similar results (FIG. 10A).

The identification of many mitotic genes as RSL candidates indicate that Ras Mut cells might experience heightened mitotic stress. Indeed, despite having a faster doubling time (FIG. 8C) and a similar percentage of G2 cells when compared to DLD-1 Ras WT cells (26.1±3.3 vs. 26.7±4.5), DLD-1 Ras mutant cells show a 50% higher mitotic index, indicative of a slower mitotic progression (FIG. 10B). Furthermore, when released from metaphase block induced by the Eg5 kinesin inhibitor monastrol (Mayer et al., 1999), a significantly higher fraction of Ras Mut cells exhibit lagging chromosome in anaphase (FIG. 10C). To further explore the consequences of mitotic stress pharmacologically, the sensitivity of the Ras Mut and WT cells towards two inhibitors of mitotic spindle function (nocodazole and paclitaxel) was determined (Peterson and Mitchison, 2002). Whereas both the Ras Mut and WT cells show comparable sensitivity to the microtubule depolymerizer nocodazole (FIG. 17A), both DLD-1 and HCT116 Ras Mut cells show increased sensitivity to the microtubule stabilizer paclitaxel relative to their respective WT counterparts (FIG. 10D and FIG. 17B). Cell cycle analysis reveals that, at the synthetic lethal concentration of paclitaxel causes a strong G2/M arrest in Ras Mut cells but not in Ras WT cells that is attributed to a striking pro-metaphase block in the Ras mutant cells (FIGS. 10 E&F). Together these findings indicate that the Ras oncogene causes increased mitotic stress and renders the cell hypersensitive to perturbation of the mitotic machinery.

### Ras Mutant Cells are Hypersensitive to Inhibition of PLK1 Function

Polo-like kinase 1 (PLK1) plays a key role in mitosis (Barr et al., 2004; Petronczki et al., 2008). Its activity is often deregulated in cancer cells and inhibitors against PLK1 have been developed as potential cancer therapeutics (Strebhardt and Ullrich, 2006). Multiple shRNAs against PLK1 were discovered to show increased toxicity towards Ras Mut cells compared to Ras WT cells in both the DLD-1 and the HCT116 isogenic pairs (FIG. 11A). Furthermore, siRNAs against PLK1 also yielded enhanced toxicity towards Ras Mut cells (FIG. 18A).

To further confirm these results, the effect of BI-2536, a highly selective small molecule inhibitor of polo-like kinases, particularly PLK1 (Steegmaier et al., 2007; Lenart et al., 2007) was tested. An increased sensitivity of the Ras Mut cells towards BI-2536 in both the DLD-1 and HCT116 isogenic pairs was observed (FIG. 11B). The synthetic lethal effect of BI-2536 can be reproduced using either the competition assay (FIG. 11B) or by measuring the viability of Ras Mut and WT cells independently (FIG. 20), and was found to be strongest at approximately 25 nM in DLD-1 cells. The cell cycle distribution of DLD-1 cells was analyzed after treatment with either 25 nM or 50 nM of BI-2536 for one day. Whereas the cell cycle profile of Ras WT cells is only modestly affected, Ras Mut cells show a profound G2/M accumulation in the presence of BI-2536 (FIG. 11C). Careful analysis reveals that this G2/M accumulation in Ras Mut cells is due to a strong block of Ras Mut cells in prometaphase: whereas a substantial number of metaphase and anaphase cells could still be found among Ras WT cells in the presence of BI-2536, few such cells were found among the Ras Mut cells (FIG. 11D).

PLK1 functions at multiple stages during mitosis from mitotic entry all the way through cytokinesis (Petronczki et al., 2008). To investigate whether PLK1 inhibition delays mitotic entry in DLD-1 Ras Mut cells, cells were synchronized at the G2/M boundary using the CDK1 inhibitor RO-3306 (Vassilev et al., 2006) and released them into nocodazole with or without the presence of BI-2536 to trap mitotic cells. It was found that mitotic entry was faster for Ras Mut cells but was unaffected at the synthetic lethal concentration of BI-2536 in either Ras WT or Ras Mut cells (FIG. 11E). Next, mitotic cells synchronized in nocodazole were released to test their ability to complete mitosis in the presence of BI-2536. Whereas BI-2536 had a minimal effect on Ras WT cells in this respect, it caused a profound delay in mitotic exit in Ras Mut cells (FIG. 11F). This finding further supports the notion that Ras Mut cells are more dependent upon PLK1 activity for mitotic progression. The mitotic arrest of Ras Mut cells in BI-2536, however, was not sustained over time. Prolonged treatment with BI-2536 for 2 days results in an elevated sub-G1 population in Ras Mut cells, indicative of cell death (FIG. 11G).

PLK1 is transcriptionally upregulated in late S and in G2 phase, whereas its catalytic activation requires phosphorylation at Thr210 by the kinase Aurora-A at the G2/M transition (Jang et al., 2002; Seki et al., 2008; Macurek et al., 2008). Without wishing to be bound by theory, one explanation of the results would be that Ras Mut cells have lower PLK1 protein levels or activity during mitosis. Contrary to this model, the levels of both total and activated PLK1 protein are slightly elevated in Ras Mut cells during mitosis, particularly at the G2/M boundary (FIG. 18G). Thus the increased dependency of Ras Mut cells on PLK1 function is likely due to other causes rather than reduced PLK1 levels in the Ras Mut cells. Together these findings indicate that the Ras oncogene adversely affects mitotic progression and renders cells more dependent on PLK1 activity for proper mitosis progression.

### Ras Mutant Cells are Hypersensitive to APC/C and Proteasome Inhibition

Mitotic progression is critically controlled by the activity of the anaphase promoting complex/cyclosome (APC/C), an E3 ubiquitin ligase that promotes the orderly degradation of key mitotic proteins (Peters, 2006). Several APC/C subunits including APC1/ANAPC1, APC4/ANAPC4, Cdc16 and Cdc27 scored in the screen (Tables 1&2, FIG. 12A), suggesting that Ras Mut cells are more dependent on APC/C activity for mitotic progression. The activity of APC/C is inhibited by EMI1 prior to mitosis, until PLK1 phosphorylates EMI1 and targets it for degradation via the SCF (Reimann et al., 2001; Hansen et al., 2004; Di Fiore and Pines, 2007). The binding partner of EMI1, EVI5, on the other hand, blocks PLK1 phosphorylation of EMI1 and thereby antagonizes APC/C activation (Eldridge et al., 2006). Thus Ras Mut cells are more dependent on APC/C activity, and are also more sensitive to EMI1 or EVI5 overexpression. When exogenous EMI1 and EVI5 were retrovirally expressed in these cells, Ras Mut cells were specifically impaired (FIG. 12B). These results indicate that either APC/C activation might be reduced in Ras Mut cells or that these cells show a higher dependence on normal APC activity for survival. Consistent with these models, sub-phenotypic siRNA knockdown of APC/C subunits strongly synergizes with sub-phenotypic low concentrations of BI-2536 to confer synthetic lethality in DLD-1 Ras Mut cells (FIG. 12C).

Two steps in the central pathway identified in the screen require proteolysis. Both activation of the APC through EMI ubiquitination and APC/C targeted ubiquitination of mitotic proteins ultimately require proteasome activity for degradation. Importantly, the screen also identified shRNAs against several proteasome subunits including PSMA5, PSMB5 and PSMB6 (FIG. 12D). Furthermore, two structurally distinct small molecule inhibitors of the proteasome, MG132- and bortezomib (Velcade), both exhibit synthetic lethality with Ras Mut cells (FIG. 12E and FIG. 20). Without wishing to be bound by theory, Ras Mut cells are more sensitive to MG132- and bortezomib-induced G2/M arrest, which is consistent with a model that the hypersensitivity of Ras Mut cells to proteasome inhibition is in part due to mitotic defects, (FIG. 12F). Again this is due to a more profound prometaphase block of the Ras Mut cells in the presence of these drugs (FIG. 12G). Together these results indicate that the Ras oncogene interferes with the function of the APC/C and renders cells sensitive to further inhibition of this complex.

### The Mitotic Machinery as an Achilles' Heel for Ras Mutant Cancer Cells

Taken together, these results indicate that cancer cells with mutant Ras experience elevated mitotic stress and are more dependent on key mitotic proteins such as PLK1, the APC/C complex, the COP9 signalosome and the proteasome for proper mitotic progression (FIG. 13), It is also shown herein that targeting selected mitotic proteins can exacerbate this mitotic stress to selectively kill Ras mutant cancer cells. It is shown herein that small molecule inhibitors, which disrupt mitosis (e.g., paclitaxel, BI-2536, bortezomb (Velcade), MG132, etc.) all constitute synthetic lethality with Ras mutant cells. Transient treatment of DLD-1 cells for 24 hours with these drugs, which approximates the length of one cell cycle for these cells, is sufficient to selectively impair the viability of Ras Mut cells (FIG. 20). This indicates that many Ras Mut cells might not recover from their drug-induced mitotic arrest and fail to complete normal mitosis following the removal of these inhibitors.

To assess whether this mitotic stress might be specifically associated with the Ras oncogene, a potential synthetic lethal effect of paclitaxel, BI-2536, bortezomib and MG132 was tested in isogenic DLD1 and HCT116 cells for the PI3K oncogene (Samuels et al., 2005). In contrast to Ras Mut DLD-1 cells, DLD-1 PI3K Mut cells are more resistant to these inhibitors relative to DLD-1 PI3K WT cells. Furthermore, HCT116 PI3K Mut and WT cells showed very little difference when treated with these inhibitors (FIG. 21). These results indicate that the increased mitotic stress observed is specific for oncogeneic Ras and not for PI3K activation.

Although the detailed mechanisms by which the Ras oncogene affects the activity of various mitotic machineries remain to be elucidated, these data indicate that impaired APC/C function might be a critical oncogenic stress associated with Ras mutation (FIG. 13). Without wishing to bound by theory, Ras mutant cancer cells can benefit from increased APC/C activity. Support for this hypothesis comes from analysis of lung cancer tumor samples. The expression of the core mitotic RSL candidate genes and other genes directly associated with APC/C function (all APC/C subunits, EMI1, EVI5, Cdc20, Cdh1 and UbCH10) was analyzed and correlated with patient prognosis in a cohort of lung cancer samples (Shedden et al., 2008). As the mutation status of the Ras genes in these tumors is currently unknown but their transcriptional profiles are known, a Ras expression signature was first derived from a separate, smaller set of lung tumors whose Ras mutation status are known (Bhattacharjee et al., 2001). This Ras signature was applied to the cohort to stratify them as having positive, negative or neutral Ras signatures (FIG. 14A). 143 tumors were defined as having a strong Ras mutant signature (Ras signature +) and 116 as having a WT-Ras signature (Ras signature −). Three genes, COPS3, Cdc16 and EVI5, showed a correlation of expression level which is associated with prognosis in a manner that is also dependent on the tumor's Ras signature status. Lower expression of COPS3 and Cdc16, and higher expression of EVI5 (all of which are consistent with potentially decreased APC/C activity) are each associated with enhanced survival for patients bearing tumors with a positive Ras signature but have no prognostic value in patients bearing tumors with a negative Ras signature (FIG. 14B). When tumors are simultaneously investigated for all three gene signatures, those with lower COPS3 and Cdc16 together with higher EVI5 expression levels are associated with an enhancement of survival for patients bearing tumors that exhibit a positive Ras signature (FIG. 14C). These findings are consistent with the hypothesis that APC/C activity could present a limiting factor in Ras mutant cancer cells and present an attractive drug target for cancers with Ras mutation.

Using this pool-based shRNA platform in a genome-wide screen, candidate Ras synthetic lethal (RSL) genes of diverse functions were identified whose knockdown constituted synthetic lethality with the KRAS oncogene. Many of these RSL genes are implicated in Ras function for the first time. This study, together with previous analyses of the Ras pathway [Reviewed in (Karnoub and Weinberg, 2008)], indicate that a broad genetic network spanning multiple cellular functions are required to support the Ras oncogenic state. Many genes in this network could be exploited as potential therapeutic targets, as demonstrated by the synthetic lethal effect of their knockdown by RNAi.

### Mitotic Stress as a Hallmark of the Ras Oncogenic State

Multiple mitotic genes were identified whose knockdown constitutes synthetic lethality with mutant Ras in DLD-1 and HCT116 colorectal cancer cells. Among these is the centromeric hMis18 complex that functions to recruit centromere-specific histone CENP-A (Fujita et al., 2007), the kinetochore protein KNL-1, a member of the KNL-1/M is12/Ndc80 complex that promotes spindle attachment and proper chromosomal segregation (Cheeseman et al., 2004; Cheeseman et al., 2006), the microtubule depolymerizing kinesin MCAK that promotes the resolution of merotelic kinetochore attachment (Andrews et al., 2004; Lan et al., 2004; Kline-Smith et al., 2004), as well as the chromosomal passenger complex (Ruchaud et al., 2007), the APC/C (Peters, 2006) and the mitotic kinase PLK1 (Barr et al., 2004, Nat Rev Mol Cell Biol, 5, 429-40; Petronczki et al., 2008, Dev Cell, 14, 646-59), all of which play a multitude of roles in mitotic progression. This indicates that the Ras oncogene induces mitotic stress and renders the cells more dependent on the function of key mitotic proteins for survival. In agreement with these findings, it has been previously shown that mutant Ras can induce chromosome instability (Denko et al., 1994), and RNAi knockdown of the chromosomal passenger complex protein survivin and the kinesin-like protein TPX2 also selectively kill Ras mutant cells (Sarthy et al., 2007; Morgan-Lappe et al., 2007). Together these findings indicate that targeting selected components of the mitotic machinery can prove useful in treating Ras-driven cancers. Indeed, the data presented herein show that several pharmacological inhibitors of mitosis selectively impair the viability of Ras mutant cells. This study identifies a previously unappreciated role of Ras in regulating mitotic progression.

Without wishing to be bound by theory, the following mechanisms are proposed. Acute expression of mutant H-Ras can induce chromosomal instability within a single cell cycle (Denko et al., 1994), thus suggesting a potentially direct mechanism. Several reports have implicated the Ras/MAPK pathway in mitosis. It has been previously shown that in yeast Ras negatively regulates the kinetochore DASH complex (Li et al., 2005) and thus might influence spindle attachment. In mitotic Xenopus oocyte extract, MAPK activity is required for mitotic entry and the maintenance of the mitotic state, whereas its inactivation is required for mitotic exit (Wang et al., 2007; Guadagno and Ferrell, 1998). In mammalian cells, MAPK activity is require for G2/M transition (Wright et al., 1999), and activated Ras can accelerate mitotic entry (Knauf et al., 2006). Activated MAP kinase localizes to the mitotic kinetochore (Zecevic et al., 1998; Shapiro et al., 1998) and a hyperactive MAPK pathway can promote spindle checkpoint bypass (Eves et al., 2006). In support of this, MAP kinase has been shown to phosphorylate the substrate-recognition subunit of APC/C, Cdc20, and release it from the spindle checkpoint complex Mad2/BubR1 (Chung and Chen, 2003). Without wishing to be bound by theory Ras/MAPK signaling might affect the fidelity of the spindle checkpoint. The finding that Ras Mut cells are hypersensitive to the knockdown of KNL-1, MCAK and to paclitaxel would support this hypothesis. However, no hypersensitivity of Ras Mut cells to nocodazole was observed, and contrary to a mitotic checkpoint by-pass phenotype, a more profound prometaphase arrest was observed in Ras Mut cells when treated with paclitaxel or BI-2536.

This study also identified a novel genetic interaction between the Ras oncogene and the APC/C. In yeast, there is evidence that Ras can negatively regulate APC/C activity (Irniger et al., 2000). In unfertilized Xenopus oocytes arrested at metaphase of meiosis II, a signaling pathway involving Mos-MAPK, p96RSK activates the APC/C inhibitor Erp1/Emi2 to inhibit APC/C activation (Inoue et al., 2007; Nishiyama et al., 2007). The mechanism by which this occurs in mammalian cells during mitosis is unknown. Importantly, this study indicates that APC/C itself might be an attractive drug target—small molecule inhibitors targeting the E3 ubiquitin ligase activity of APC/C might cause synthetic lethality either alone or in combination with PLK1 inhibitors in Ras mutant tumors.

Many RSL genes identified are growth and survival genes. It is likely that the synthetic lethality conferred by their respective shRNAs results from a partial knockdown of their function. In the case of PLK1, this is supported by the observation that its inhibitor BI-2536 exhibits Ras synthetic lethality at concentrations below that needed to cause complete mitotic arrest. Without wishing to be bound by theory, such heightened dependency of Ras mutant cells on essential gene function could therefore reflect a critical fitness cost associated with oncogenic stress. Indeed, this study demonstrates that therapeutic strategies aim at suppressing the Ras oncogenic pathway directly (e.g. RNAi against Ras), inhibiting the stress support pathways protecting the cancer cells from oncogenic stress (e.g. RNAi and inhibitors against the proteasome and the APC/C), or enhancing the stress phenotype of cancer cells (e.g. paclitaxel) could all selectively impair the viability of Ras mutant cancer cells.

The approach described herein enables the rapid identification of functional vulnerabilities in cancer cells for therapeutic exploitation. Based on this genetic analysis, a number of small molecule inhibitors have been identified to be potentially useful for treating tumors with Ras mutations. Two of these, the microtubule stabilizer paclitaxel and the proteasome inhibitor bortezomib, have already been approved for the treatment of certain types of cancers. The third agent, the PLK1 inhibitor BI-2536, is currently undergoing phase I clinical trials (Mross et al., 2008).

The present study illustrates the potential rapidity by which synthetic lethality analysis could be translated into better therapeutic strategies. Complementary to the physical mapping of cancer genomes, functional approaches such as RNAi screen can identify genetic dependencies of cancer cells regardless of the mutational status of the gene of interest. Indeed, many candidate RSL genes identified have not been found to be mutated in tumors and are unlikely to be oncogenes themselves. The concept of “non-oncogene addiction” has recently been proposed to describe the extensive dependency of cancer cells on the function of diverse networks of genes—many of which are neither mutated or oncogenic—for their growth and survival (Solimini et al., 2007). This study provides a glimpse of the landscape of non-oncogene addiction and indicates this is an area that is likely to shed new light on the mechanisms of tumorigenesis and presents new opportunities for cancer therapeutics.

### Materials and Methods

shRNA Library Screen and Microarray Hybridization

The pool-based shRNA screen using half-hairpin (HH) barcode deconvolution was carried out as described before (Schlabach et al., 2008). Briefly, DLD-1 Ras mutant (Mut) or wildtype (WT) cells were infected with pools of retroviral shRNA at a representation of ˜1,000 and a multiplicity of infection (MOI) of ˜1. At day 3 post infection an initial, population-doubling 0 (PD0) sample was taken. The rest of the population were selected with puromycin to remove the minority of uninfected cells and propagated in culture for an additional 17 doublings before the final, PD17 sample was taken. For each passage a minimal representation of 1000 was maintained. The genomic shRNA library containing 74,905 retroviral shRNAs targeting 32,293 unique human gene transcripts (including 19,542 RefSeqs) were screened as 6 pools of ˜13,000 shRNAs per pool in independent triplicates. For each pair of corresponding PD0 and PD17 samples, shRNA HH barcode was PCR-recovered from genomic samples and labeled with Cy5 and Cy3 dyes, respectively. The labeled HH barcode amplicons were competitively hybridized to a microarray containing the corresponding probes. Custom microarrays with HH barcode probe sequences were from Roche Nimblegen. Array hybridization and scanning protocols were based on manufacturer's instructions. The genome-wide mir30 shRNA library was expressed using the retroviral vector MSCV-PM (Schlabach et al., 2008) and is available through Open Biosystems Inc.

Statistical and Bioinformatics Analysis of shRNA Screen

Only informative probes (i.e. those with raw signal 2-fold above negative control probes) were used for analysis. The change in the relative abundance of each shRNA in the library over time is measured using the normalized Cy3/Cy5 ratio of its probe signal (Schlabach et al., 2008). A log₂ Cy3/Cy5 ratio of <0 indicates the shRNA is depleted in the population over time. To identify shRNAs that are synthetically lethal with mutant Ras, the mean log₂ Cy3/Cy5 ratios of the Ras Mut triplicates were compared to that of the Ras WT triplicates to derive the log₂ ratio difference. A p-value of the difference between the two triplicates was calculated using the t-test. A extended list of candidate shRNAs (Table 1) were obtained by using a set of cutoff that requires the Ras Mut log 2 ratio≦−0.7, Ras WT log ratio≧−2, the difference in log₂ ratios between Ras Mut and Ras WT to be ≦−0.7, and the p-value to be ≦0.3. By reducing the p-value to ≦0.1 a shorter candidate list is obtained (Table 2). As the overall validation rate is relatively low (26.7%), the statistical analysis was used mainly as a guide to prioritize candidates for further test and validation. Functional categorization of candidate RSL genes was done using PANTHER (Thomas et al., 2003).

### Analysis of Human Lung Tumor Profiles

A gene signature of KRAS mutant versus KRAS wild-type tumors was defined, using a published dataset of 84 lung adenocarcimos from a study by Bhattacharjee et al. (Bhattacharjee et al., 2001) for which the KRAS mutation status of each tumor was known (genes with P<0.01, two-sided t-test were selected). This KRAS gene signature was applied to analyze a gene expression profile dataset of 442 human lung adenocarcinomas by Shedden et al. (Shedden et al., 2008). Each tumor was scored for manifestation of the Ras pathway by the following. The Shedden tumor profiles were generated among four laboratories, and so within each laboratory subset, expression values for each gene were normalized to standard deviations from the mean. Within each Shedden tumor profile, the average of the genes high (“up”) in the Bhattacharjee KRAS signature were compared with the average of the genes low (“down”) in the signature: tumors with higher expression of the “up” genes as compared to the “down” genes (P<0.01, t-test) were classified as showing Ras pathway manifestation (“Ras signature+”); tumors with higher expression of the “down” genes (P<0.01) were classified as not showing Ras pathway manifestation (“Ras signature-”); tumors that were intermediate between the above two groups were not used in subsequent analyses.

Within the Shedden “Ras signature+” tumors (N=143), tumors with expression levels greater than the median for the given candidate RSL gene were compared with the rest of the tumors in the subset, using Kaplan-Meier analysis for time to patient death. In addition, univariate Cox analysis evaluated the expression of the given gene as a continuous variable for correlation with outcome. The same Kaplan-Meier and Cox analyses were also carried out using the Shedden “Ras signature-” tumors (N=116).

### Competition Assay

The competition assay used to test candidate genes from the screen was modified from a previous protocol (Torrance et al., 2001; Smogorzewska et al., 2007) and was carried out in 96-well plates in independent triplicates. For this assay, 1,000 each of GFP-labeled Ras Mut and unlabeled Ras WT cells were seeded in each well. For retroviral shRNA infection, cells were infected at an MOI of 1-5, selected for 3 days with puromycin and propagated for an additional 4-5 days before analyzed by FACS. An shRNA targeting firefly luciferase (shRNA-FF) was used as the negative control. For siRNA transfection, cells were transfected with siGenome siRNAs (Dharmacon) using Lipofectamine RNAiMAX (InVitrogen) and analyzed by FACS 5-6 days post transfection An siRNA targeting luciferase (Luc) was used as the negative control. For drug treatment, cells were treated with drugs for 4-5 days prior to FACS analysis. Untreated wells were used as negative controls. To obtain normalized fitness of Ras Mut cells that can be compared across experiments, the percentage of Ras Mut cells in each sample was normalized to that in the control samples of that experiment. This results in a “normalized mutant fitness” that ranges from 0% (no Ras mut cells left in population) to 100% (same number of Ras mut cells as control wells).

### Cell Cycle Analysis

For cell cycle analysis, cells were ethanol fixed and stained with PI and analyzed using a BD LSR II FACS analyzer (BD Bioscience). Data analysis was carried out with BD FACS Diva and FlowJo softwares. Cell cycle synchronization by double thymidine block was based on a previous protocol (Steegmaier et al., 2007). Briefly, cells were arrested with two rounds of 20-hour thymidine (2.5 mM) treatment approximately 12 hours apart and released into mitosis. To trap cells at different stages of mitosis, cells release from double thymidine block were released into the CDK1 inhibitor RO-3306 (10 μM) (Vassilev et al., 2006), the PLK1 inhibitor BI-2536 (100 nM) (Steegmaier et al., 2007) and the microtubule depolymerizer nocodazle (100 ng/ml). For one-step arrest in G2/M or in prometaphase, cells were treated overnight with either RO-3306 (10 μM) or the Eg5 kinesin inhibitor monastrol (100 μM), respectively. For mitotic entry experiments, cells were synchronized in RO-3306 (10 uM) overnight prior to release. For mitotic release experiments, cells were released from overnight synchronization in RO-3306 (10 uM) into nocodazole (200 ug/ml) for 4 hours and mitotic cells were collected by mitotic shake-off. Cells were then release by washing out the nocodazole. For the analysis of lagging chromosomes and mitotic index by microscopy, cells seeded onto coverslips were arrested in mitosis with 100 μM monastrol for 16 hours. Cells were washed and released into fresh media and were fixed at ten minute intervals for two hours in PHEM buffer+2% formaldehyde+0.5% triton-X100. Cells were immunostained with DAPI to visualized DNA and antibodies to phospho-histone H3 Ser10 (pH3S10) and to α-tubulin to identify mitotic cells at different stages. Independent triplicates with >100 cells per replicate were analyzed. For measuring mitotic index by FACS, cells were trypsinized, fixed with ethanol and stained with PI and pH3S10 antibody.

### Cell Culture, Molecular Biology and Reagents

The DLD-1 and HCT116 isogenic cells were kind gifts from Dr. Bert Vogelstein (Johns Hopkins University School of Medicine) and were maintained in McCoy's 5A media supplemented with 10% FBS and antibiotics. For proliferation assays, 10,000 cells were seeded in each well of a 24-well plate and cell number was measured using CellTiterGLO (Promega). For adherent colony assays, 1000 cells were seeded in each well of a 6-well plate and colonies were counted 10 days later by Coomassie staining. For anchorage independent colony assays, 1000 cells were seeded in each well of a 6-well plate in media containing 0.35% low-melting point agarose and colonies were counted 3 weeks later by crystal violet staining. The PCR primers used to amplify HH barcodes from genomic DNA samples are JH353F 5′-TAGTGAAGCCACAGATGTA-3′ and HHR2L 5′-ATGTATCAAAGAGATAGCAAGGTATTCAG-3′. BI-2536 was a kind gift from Dr. Nathanael Gray (Harvard Medical School and Dana Faber Cancer Institute). Paclitaxel, nocodazole, MG132 and monastrol were from Sigma-Aldrich. RO-3306 was from EMD Calbiochem. Bortezomib was from LC Laboratories. Rabbit antibody against phospho-PLK1 T210 was from Abcam. Rabbit phosphoH3-S10 antibody was from Covance. Mouse antibodies against PLK1, K-Ras and tubulin were from Santa Cruz Biotechnologies. Rabbit anti-COPS4 antibody was from Bethyl Laboratories.

### REFERENCES

S1. C. Lee, J. S. Kim, T. Waldman, Cancer Res. 64, 6906 (2004).
S2. G. Keller, M. Kennedy, T. Papayannopoulou, M. V. Wiles, Mol. Cell. Biol. 13, 473 (1993).
S3. E. Robertson, A. Bradley, M. Kuehn, M. Evans, Nature 323, 445 (1986).
S4. M. Z. Li, S. J. Elledge, Nat. Genet. 37, 311 (2005).
S5. V. G. Tusher, R. Tibshirani, G. Chu, Proc. Natl. Acad. Sci. USA 98, 5116 (2001).

Lengthy table referenced here
US20110081362A1-20110407-T00001
Please refer to the end of the specification for access instructions.

Lengthy table referenced here
US20110081362A1-20110407-T00002
Please refer to the end of the specification for access instructions.

Lengthy table referenced here
US20110081362A1-20110407-T00003
Please refer to the end of the specification for access instructions.

Lengthy table referenced here
US20110081362A1-20110407-T00004
Please refer to the end of the specification for access instructions.

Lengthy table referenced here
US20110081362A1-20110407-T00005
Please refer to the end of the specification for access instructions.

Lengthy table referenced here
US20110081362A1-20110407-T00006
Please refer to the end of the specification for access instructions.

Lengthy table referenced here
US20110081362A1-20110407-T00007
Please refer to the end of the specification for access instructions.

Lengthy table referenced here
US20110081362A1-20110407-T00008
Please refer to the end of the specification for access instructions.

Lengthy table referenced here
US20110081362A1-20110407-T00009
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110081362A1). An electronic copy of the table will also be available from the USPTO upon request and payment of the fee set forth in 37 CFR 1.19(b)(3).

53. A method for selectively inhibiting growth or survival of a cancer cell bearing an activating Ras mutation in an individual, the method comprising:
a. determining whether cancer cells of said individual bear an activating Ras mutation; and, if so,
b. contacting said cancer cell with an inhibitor that targets a regulator of a cellular process or a cellular component selected from the group consisting of mitosis and chromosomal segregation, proteasomes, COPS signalosome, protein translation, ubiquitination, protein neddylation, sumoylation, and RNA splicing,
wherein the regulator is a gene product selected from the group consisting of cyclin A2 (CCNA2), hMIS18α, hMIS18β, (C21ORF45, OIP5), borealin (CDCA89), KNL-1 (CASC5), MCAK (KIF2C), subunits of the APC/C complex (ANAPC1, ANAPC4, CDC16, CDC27), SMC4, PIK-1, PSMA5, PSMB5, PSMB6, BUB1, CAND1, CLDN1, COPS3, F8, FABP3, FBXO3, HGS, JAKE KCNA3, LMTK3, MAP4K4, MAPK1, MYST3, PI4K2B, PKN1, PPP1R10, PRKCB1, PTPRE, RAD51C, RAFT, RNF20, SENP1, SENP8, SP100, TDRD3, TERF1, TRIM54, UCKL1, XPO1, BXDC2, FBL, NOL5A, EIF3S8, EIF3S4, GSPT1, HNRNPC, METAP1, COPS4, COPSE, NEDD8, NAE1/APPBP1, SAE1, UBA2, UBE21, FIP1L1, NXF1, USP39, DHX8, and THOC1.
wherein said inhibitor reduces growth or survival of said cancer cell by disrupting mitosis in said cancer cell and
wherein said inhibitor is selectively toxic to said cancer cell as compared to a normal cell.

54. The method of claim 53, wherein said inhibitor is selected from the group consisting of an RNA interference molecule, a small molecule, an antibody, an aptamer and a nucleic acid.

55. The method of claim 53, said contacting steps comprises treating said cancer cell with an inhibitor of a plurality of said genes.

56. The method of claim 53, further comprising contacting with a chemotherapeutic agent for combination therapy.

57. The method of claim 53, wherein said inhibitor is a small molecule selected from the group consisting of paclitaxel, nocodazole, monastrol, BI2536, MG132, or bortezomib.

58. A method for treating cancer in an individual, the method comprising:
c. determining whether cancer cells of said individual bear an activating Ras mutation; and, if so,
d. administering to said individual an inhibitor that targets a regulator of a cellular process or a cellular component selected from the group consisting of mitosis and chromosomal segregation, proteasomes, COPS signalosome, protein translation, ubiquitination, protein neddylation, sumoylation, and RNA splicing,
wherein the regulator is a gene product selected from the group consisting of cyclin A2 (CCNA2), hMIS18a, hMIS18β, (C21ORF45, OIP5), borealin (CDCA89), KNL-1 (CASC5), MCAK (KIF2C), subunits of the APC/C complex (ANAPC1, ANAPC4, CDC16, CDC27), SMC4, PIK-1, PSMA5, PSMB5, PSMB6, BUB1, CAND1, CLDN1, COPS3, F8, FABP3, FBXO3, HGS, JAKE KCNA3, LMTK3, MAP4K4, MAPK1, MYST3, PI4K2B, PKN1, PPP1R10, PRKCB1, PTPRE, RAD51C, RAFT, RNF20, SENP1, SENP8, SP100, TDRD3, TERF1, TRIM54, UCKL1, XPO1, BXDC2, FBL, NOL5A, EIF3S8, EIF3S4, GSPT1, HNRNPC, METAP1, COPS4, COPSE, NEDD8, NAE1/APPBP1, SAE1, UBA2, UBE2I, FIP1L1, NXF1, USP39, DHX8, and THOC1.
wherein said inhibitor reduces growth or survival of said cancer cell by disrupting mitosis in said cancer cell and
wherein said inhibitor is selectively toxic to said cancer cell as compared to a normal cell.

59. The method of claim 58, wherein said inhibitor is selected from the group consisting of an RNA interference molecule, a small molecule, an antibody, an aptamer and a nucleic acid.

60. The method of claim 58, said contacting steps comprises treating said cancer cell with an inhibitor of a plurality of said genes.

61. The method of claim 58, further comprising contacting with a chemotherapeutic agent for combination therapy.

62. The method of claim 58, wherein said inhibitor is a small molecule selected from the group consisting of paclitaxel, nocodazole, monastrol, BI 2536, MG132, or bortezomib.

63. A method for determining prognosis in an individual having an activating Ras mutation, the method comprising:
a. measuring the levels of COPS3, Cdc16, and EV15 in a test sample from an individual having an activating Ras mutation; and
b. comparing the levels of COPS3, Cdc16 and EV15 to the levels of COPS3, Cdc16, and EV 15 in a reference sample,
wherein a decreased level of COPS3, a decreased level of Cdc16 and an increased level of EV15 compared to said reference sample indicates a positive prognosis, and
wherein a larger degree of change indicates a more positive prognosis.

64. The method of claim 63, wherein said test sample is a biopsy sample.

65. The method of claim 63, wherein said reference sample is obtained from said individual.

66. The method of claim 63, wherein said reference sample is obtained from said individual prior to onset of a detectable cancer.

67. The method of claim 63, wherein said reference sample is obtained from a non-cancerous tissue.

68. The method of claim 63, wherein said reference sample is obtained from a population of individuals.
